(19) United States
(12) Patent Application Publication
(10) Pub. No.: US 2023/0272462 A1
(43) Pub. Date: Aug. 31, 2023

(54) KITS, METHODS, POLYPEPTIDES, SYSTEMS, AND NON-TRANSITORY, MACHINE-READABLE STORAGE MEDIA FOR DETECTING A NUCLEIC ACID

(71) Applicant: UNIVERSITY OF WASHINGTON, Seattle, WA (US)

(72) Inventors: Enos Kline, Seattle, WA (US); Michael Roller, Seattle, WA (US); Robert G. Atkinson, Seattle, WA (US); Nuttada Panpradist, Seattle, WA (US); Daniel Leon, Seattle, WA (US); Ian Hull, Seattle, WA (US); Qin Wang, Seattle, WA (US); Barry R. Lutz, Seattle, WA (US)

(73) Assignee: UNIVERSITY OF WASHINGTON, Seattle, WA (US)

(21) Appl. No.: 18/004,489

(22) PCT Filed: Jul. 9, 2021
(86) PCT No.: PCT/US2021/041035

§ 371 Date: Jan. 6, 2023

Related U.S. Application Data
(60) Provisional application No. 63/049,758, filed on Jul. 9, 2020.
(60) Provisional application No. 63/049,941, filed on Jul. 9, 2020.
(60) Provisional application No. 63/050,022, filed on Jul. 9, 2020.
(60) Provisional application No. 63/050,031, filed on Jul. 9, 2020.

(51) Int. Cl. C12Q 1/6844; C12Q 1/6883; C12N 15/63; B01L 7/00

(52) U.S. Cl. CPC C12Q 1/6844; C12Q 1/6883; C12N 15/63; B01L 7/52; C12N 2310/122; B01L 2300/1822

(57) ABSTRACT

Kits, methods, polypeptides, systems, and non-transitory, machine-readable storage media for detecting a nucleic acid in a sample are described. In an embodiment, the kit comprises a loop primer nucleic acid molecule configured for loop-mediated isothermal amplification (LAMP), the loop primer nucleic acid molecule comprising: a targeting sequence complementary to a target portion of a target nucleic acid sequence; and an adapter sequence; a displacement nucleic acid probe comprising: a fluorophore adapter sequence; and the adapter sequence; and a fluorophore adapter complement nucleic acid molecule complementary to the fluorophore adapter sequence, wherein the fluorophore adapter sequence or the fluorophore adapter complement nucleic acid molecule is coupled to a fluorophore. In an embodiment, the system comprises a thermal subsystem for heating a sample disposed therein, and an optical subsystem for optically excited the sample and detecting light emitted from the sample.

### CROSS-REFERENCES TO RELATED APPLICATIONS

The present application claims the benefit of co-pending U.S. Provisional Patent Application No. 63/049,758, filed Jul. 9, 2020; co-pending U.S. Provisional Patent Application No. 63/049,941, filed Jul. 9, 2020; co-pending U.S. Provisional Patent Application No. 63/050,022, filed Jul. 9, 2020; and co-pending U.S. Provisional Patent Application No. 63/050,031, filed Jul. 9, 2020; each of which is incorporated by reference in its entirety.

### STATEMENT AS TO FEDERALLY SPONSORED RESEARCH

This invention was made with government support under Grant Nos. R01 AI140845, R61 AI140460, and T32 GM008268 awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1128WOUW_Seq_List_FINAL_20210707_ST25.txt. The text file is 19 KB; was created on Jul. 7, 2021; and is being submitted via EFS-Web with the filing of the specification.

### BACKGROUND

The COVID-19 pandemic is an unprecedented crisis in the modern era, spreading across the planet in a matter of months sickening millions and killing millions, and disrupting the lives of billions. An essential element of the response strategies to COVID-19 is diagnostic testing, which informs clinical intervention, quarantine, and epidemiological monitoring. Nucleic acid amplification tests (NAATs) remain the most accurate approach for diagnosis of infectious diseases including SARS-CoV-2 infection. However, RNA viruses like SARS-CoV-2 have a high mutational rate, which can result in elevated levels of sequence diversity accumulating as they propagate. This is a critical obstacle for NAATs because mismatches between the primer oligonucleotides and the template sequences can impair an assay and produce false negative results. As transmission has progressed, SARS-CoV-2 has diversified in distinct lineages, each with signature mutations throughout the genome. The emergence of this genetic diversity has rendered some NAATs susceptible to false negative results, causing these tests to be altered or withdrawn by the U.S. FDA. This challenge posed by mutation for NAATs is not limited to SARS-CoV-2; similar phenomena have been observed for other human pathogens.

Laboratory testing strategies to lessen this risk include redundant testing with alternative methods, diagnostic panels with multiple target regions, and/or primer sets with degenerate bases to account for known genetic variability. While degenerate primers are accessible and inexpensive, they are often limited by assay design constraints and do not account for unknown or novel mutations. Repeat and multiple testing is an effective strategy, but requires additional resources, labor, and complexity of design or implementation. These considerations are manageable in contemporary diagnostic laboratories but can be prohibitive in lower resource settings. Nearly all laboratory assays for SARS-CoV-2 use redundant targets to mitigate mutations and an internal control to account for sample processing or interference.

A critical aspect of the Center for Disease Control and Prevention's (CDC) Strategy for Global Response to COVID-19 is augmenting our current ability to rapidly identify COVID-19 infections so that the chain of transmission can be disrupted. Essential to this effort is the development of diagnostics that can be performed at the point-of-care (POC); that minimize the time to result (TTR) of the test and are deployable in otherwise underserved populations. These settings are inherently “low resource”, and necessitate diagnostic methods with simplified chemistry, hardware, and limited sample processing relative to the standard of practice for molecular diagnostics, polymerase chain reaction (PCR). Advancements in isothermal nucleic acid amplification technologies over the past three decades largely satisfy these constraints while still providing high sensitivity. This has led to a boom in isothermal amplification technologies and NAATs based on them. Despite their advantages, there are some areas where the isothermal NAATs are lacking when compared to PCR. Single-pot multiplexing has been infrequently demonstrated despite being a prerequisite for internal amplification control (IAC) systems and useful for multiple target redundancy.

### SUMMARY

To address these and related challenges, in certain aspects, the present disclosure provides kits and methods of detecting a presence or absence of a target nucleic acid sequence in a sample, as well as a polypeptide for use in such methods and kits. In particular and in certain embodiments, the present disclosure provides a multiplexed reverse transcriptase LAMP (mRT-LAMP) combining three assays, each targeting a unique region of the nucleocapsid (NC) gene, and an IAC assay to validate diagnostic viability with a negative result. Additionally, to accomplish this, the present disclosure provides in, certain embodiments, a universal target specific fluorescence probe system. In this regard, engineered adapter sequences are incorporated into the LAMP amplicons which then serve as a template for detection by displacement probes. The resulting assay chemistry is sensitive, specific, and durable while simplifying the development process.

Accordingly, in an aspect, the present disclosure provides a kit for detecting a presence or absence of a target nucleic acid sequence in a sample. In an embodiment, the kit comprises a loop primer nucleic acid molecule configured for loop-mediated isothermal amplification (LAMP), the loop primer nucleic acid molecule comprising: a targeting sequence complementary to a target portion of a target nucleic acid sequence; and an adapter sequence; a displacement nucleic acid probe comprising: a fluorophore adapter sequence; and the adapter sequence; and a fluorophore adapter complement nucleic acid molecule complementary to the fluorophore adapter sequence, wherein the fluorophore adapter sequence or the fluorophore adapter complement nucleic acid molecule is coupled to a fluorophore.

In another aspect, the present disclosure provides a method of detecting a presence or absence of a target nucleic acid sequence in a sample, the method comprising: contacting the sample with reagents comprising: a loop primer nucleic acid molecule configured for LAMP, the loop primer nucleic acid molecule comprising: a targeting sequence complementary to a target portion of a target nucleic acid sequence; and an adapter sequence; a displacement nucleic acid probe comprising: a fluorophore adapter sequence; and the adapter sequence; and a fluorophore adapter complement nucleic acid molecule complementary to the fluorophore adapter sequence, wherein the fluorophore adapter sequence or the fluorophore adapter complement nucleic acid molecule is coupled to a fluorophore maintaining the sample and the reagents under conditions and for a time sufficient to amplify nucleic acid molecules comprising the target nucleic acid sequence; and detecting the presence or absence of fluorescence from the fluorophore.

In another aspect, the present disclosure provides a polypeptide comprising an amino acid sequence at least 55% identical to SEQ ID NO. 25.

In another aspect, the present disclosure provides a nucleic acid encoding the polypeptide according to any of the embodiments of the present disclosure.

In an aspect, the present disclosure provides a nucleic acid expression vector comprising a nucleic according to any embodiments of the present disclosure.

In an aspect, the present disclosure provides a recombinant host cell comprising a nucleic acid expression vector according to any embodiment of the present disclosure.

In another embodiment, the present disclosure provides a method of amplifying a nucleic acid molecule, the method comprising: contacting the nucleic acid molecule with a polypeptide according to any embodiment of the present disclosure under conditions and for a time sufficient to amplify the nucleic acid molecule.

In yet another embodiment, the present disclosure provides a system for detection of amplification of a nucleic acid molecule in a sample. In an embodiment, the system comprises a thermal subsystem comprising: a thermally conductive heat block defining a chamber shaped to carry a sample holder configured to carry the sample; a first aperture disposed on a first side of the heat block and positioned to emit first signal light from within the sample holder disposed in the chamber; and a second aperture disposed on a second side of the heat block opposite the first side and positioned to emit second signal light from within the sample holder disposed in the chamber; and a heat source thermally coupled to the heat block; and an optical subsystem comprising: a light source configured to emit excitation light into the chamber; a first photodetector positioned to receive the first signal light from within the sample holder through the first aperture; and a second photodetector positioned to receive the second signal light from within the sample holder through the second aperture.

In another aspect, the present disclosure provides a method of detecting amplification of a nucleic acid molecule in a sample, the method comprising: heating a thermally conductive heat block with a heat source thermally coupled to the heat block, wherein the heat block defines a chamber shaped to carry a sample holder carrying the sample; a first aperture disposed on a first side of the heat block and positioned to emit first signal light from within the sample holder disposed in the chamber; and a second aperture disposed on a second side of the heat block opposite the first side and positioned to emit second signal light from within the sample holder disposed in the chamber; emitting, with the light source, the excitation light into the sample holder; generating a first sample signal with a first photodetector based on the first signal light received by the first photodetector through the first aperture; and generating a second sample signal with a second photodetector based on the second signal light received by the second photodetector through the second aperture.

In yet another aspect, the present disclosure provides a non-transitory, machine-readable storage medium having instructions stored thereon, which when executed by a processing system, cause the processing system to perform operations including: heating a thermally conductive heat block with a heat source thermally coupled to the heat block, wherein the heat block defines a chamber shaped to carry a sample holder carrying a sample; a first aperture disposed on a first side of the heat block and positioned to emit first signal light from within the sample holder; and a second aperture disposed on a second side of the heat block opposite the first side and positioned to emit second signal light from within the sample holder; emitting, with a light source, excitation light into the sample holder disposed in the chamber; generating a first sample signal with a first photodetector based on the first signal light received by the first photodetector through the first aperture; and generating a second sample signal with a second photodetector based on the second signal light received by the second photodetector through the second aperture.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A schematically illustrates components of a kit in accordance with an embodiment of the present disclosure;

FIG. 1B schematically illustrates amplification of a target nucleic acid molecule using the kit of FIG. 1A; and

FIG. 1C graphically illustrates copies of synthetic RNA as a function of time amplified using a kit according to an embodiment of the present disclosure.

FIGS. 2A and 2B schematically illustrate multiplexed RT-LAMP (mRT-LAMP) fluorescence detection by Universal Displacement Probes (UDP). 2A) schematically illustrates UDP incorporation during LAMP amplification and activation by displacement of quenching strand. Primer and probe refer to loop (L), adapter (A), and quencher (Q), with complementary sequences denoted with the suffix “c” (e.g., “Lc” is the reverse complement “L”). 2B) schematically illustrates two-channel fluorescence detection of multiplexed redundant LAMP products (6-FAM) and shared-primer IAC (TEX615) by UDPs. Primer designations refer to forward (F), backward (B), and loop (L) using conventional LAMP terminology;

FIGS. 3A-3C illustrate analytical performance of mRT-LAMP for SARS-CoV-2. 3A) Characteristic amplification of multiplexed SARS CoV-2 target and internal amplification control (IAC) with real-time fluorescence detection by universal displacement probes (UDP). Single representative run with 200 copies of synthetic RNA input or a no template control (NTC). 3B) Analytical sensitivity of multiplexed SARS-CoV-2 target and IAC. IAC amplifications (bottom) correspond to target amplifications (top). Target synthetic RNA input: 2,000 (n=3), 200 (n=3), 20 (n=3), 10 (n=3), or 5 copies per reaction (n=4); and NTC (n=3). 3C) Time to detect signals from SARS-CoV-2 and IAC for reactions from FIG. 3B;

FIGS. 4A and 4B illustrate mRT-LAMP amplification of extracted nasal specimens. Samples confirmed positive or negative for SARS-CoV-2 and positive for the RNase P human marker by RT-PCR panel (N1, N2, RP) were amplified by duplicate mRT-LAMP reactions. Detected mRT-LAMP signals for SARS-CoV-2 (CoV) are shown in solid dots, and IAC signals are shown in open dots; replicate pairs are connected by a line segment. Mean copy number was derived from qPCR results of N1, N1 PCR;

FIGS. 5A and 5B show validation of individual SARS-CoV-2 assays. 5A) The assay was tested with synthetic RNA fragments containing the NC1 sequence, NC2 sequence, or the NC3 sequence as separate fragments. 5B) Time for detection of SARS-CoV-2 signals for the assay with individual target inputs from the plots in 5A;

FIGS. 6A-6D illustrate amplification of mock samples with sample transport medium: 6A) Water control (25% final) 6B) DMEM (25% final). 6C) VTM (25% final). 6D) NS (25% final);

FIG. 7A is an isometric view of a system, in accordance with an embodiment of the present disclosure;

FIG. 7B is another isometric view of the system of FIG. 7A, in accordance with an embodiment of the present disclosure;

FIG. 7C is a view in cross-section of the system of FIG. 7A, in accordance with an embodiment of the present disclosure;

FIG. 7D is another view in cross-section of the system of FIG. 7A, in accordance with an embodiment of the present disclosure;

FIG. 7E is an isometric view of the system of FIG. 7A with a partial cut-away, in accordance with an embodiment of the present disclosure;

FIG. 7F is an isometric view of a heat source of the system of FIG. 7A, in accordance with an embodiment of the present disclosure;

FIG. 7G is an isometric view of an optical subsystem controller of the system of FIG. 7A, in accordance with an embodiment of the present disclosure;

FIG. 8A is a partial isometric view of another system, accordance with an embodiment of the present disclosure; and

FIG. 8B is another isometric view of the system of FIG. 8A, in accordance with an embodiment of the present disclosure.

### DETAILED DESCRIPTION

Embodiments of kits, methods, polypeptides, systems and non-transitory, machine-readable storage media for detecting a nucleic acid in a sample are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Kits

In an aspect, the present disclosure provides a kit suitable for isothermal amplification of target nucleic acid molecules, such as by loop-mediated isothermal amplification (LAMP). In an embodiment, the kit comprises a loop primer nucleic acid molecule configured for loop-mediated amplification, the loop primer nucleic acid molecule comprising: a target portion complementary to a target nucleic acid sequence; and an adapter sequence; a displacement nucleic acid probe comprising: a fluorophore adapter sequence; and the adapter sequence; and a fluorophore adapter complement nucleic acid molecule complementary to the fluorophore adapter sequence, wherein the fluorophore adapter sequence or the fluorophore adapter complement nucleic acid molecule is coupled to a fluorophore.

As above, in an embodiment, the loop primer and the displacement nucleic acid probe include the adapter sequence. In an embodiment, the adapter sequence is suitable to serve as a target for the displacement nucleic acid probe, rather than being a direct modification of loop primer nucleic acid molecule target portion directly, as is the case with assimilating probes. As a consequence, in an embodiment, the displacement nucleic acid probe is entirely artificial and with no part sharing sequence identity that is endogenous to the target nucleic acid sequence.

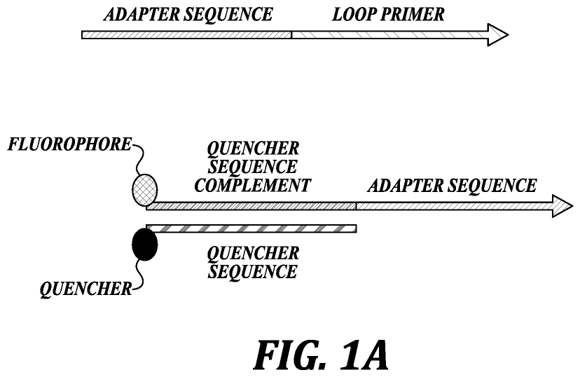

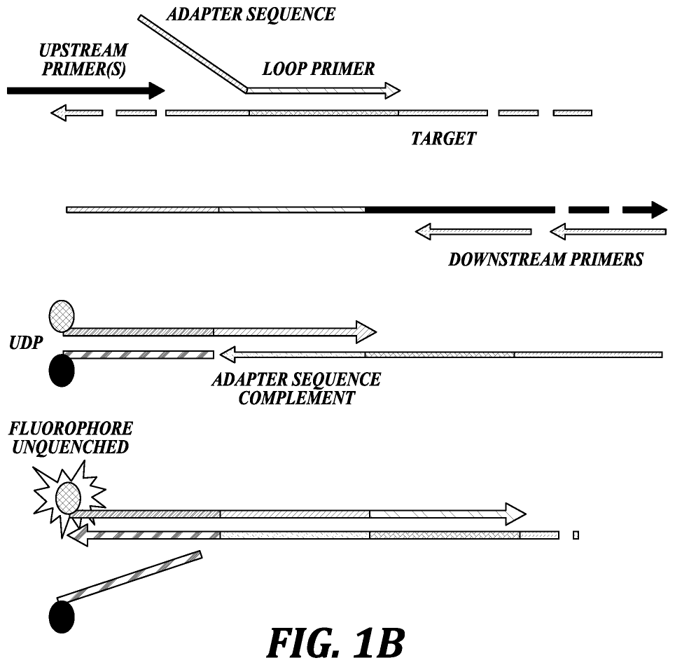

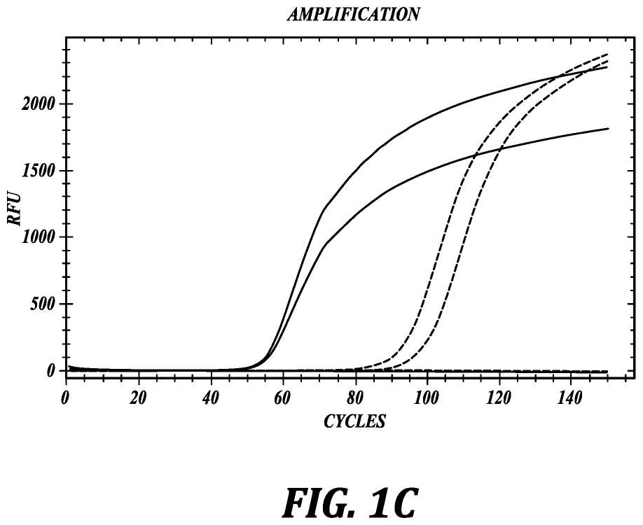

FIGS. 1A and 1B illustrate a kit according to an embodiment of the present disclosure and amplification using that kit, respectively. As shown in FIG. 1A, the kit includes a loop primer nucleic acid molecule with an adapter sequence (top) and a fluorescent displacement nucleic acid probe (bottom) including an adapter sequence on the 3′ end, and a fluorophore adapter sequence on the 5′ end of the strand, with a fluorophore attachment on the 5′ terminus; hybridized to a fluorophore adapter complement nucleic acid molecule with a quencher molecule at the 3′ end, such that the fluorophore and quencher are in close proximity when the displacement nucleic acid probe and the fluorophore adapter complement nucleic acid molecule are hybridized.

As shown in FIG. 1B, in use the fluorophore adapter complement nucleic acid molecule is displaced from the displacement nucleic acid probe resulting in un-quenching of the fluorophore and enabling fluorescence detection. Briefly, the loop primer nucleic acid molecule binds to a lengthening concatemer in a single stranded loop region and extends. This extension product is them displaced by upstream primer binding processes, as is typical in LAMP. This extended strand acts as a template for binding and extension by downstream primers (not shown), ultimately resulting in the production of a single stranded fragment with a terminal region that is complementary to the adapter sequence. This complementary sequence is then bound by the displacement nucleic acid probe, which extends, and acts as a primer extending on to the displacement nucleic acid probe displacing the fluorophore adapter complement nucleic acid molecule.

As shown, the displacement nucleic acid probe is coupled to a fluorophore and the fluorophore adapter complement nucleic acid molecule is coupled to a quencher configured to quench fluorescence of the fluorophore. It will be understood that other configuration within the scope of the present disclosure are possible. In an embodiment, wherein whichever of the fluorophore adapter sequence or the fluorophore adapter complement nucleic acid molecule is not coupled to the fluorophore is coupled to a quencher configured to quench fluorescence of the fluorophore. In an embodiment, whichever of the fluorophore adapter sequence or the fluorophore adapter complement nucleic acid molecule is not coupled to the fluorophore is coupled to a second fluorophore configured to receive energy from the fluorophore by Förster resonance energy transfer. In an embodiment, the fluorophore adapter sequence is configured to form a hairpin structure and further comprises a quencher positioned to be proximal to the fluorophore when the fluorophore adapter sequence is in the hairpin structure.

In an embodiment, the kit includes additional loop primer nucleic acid molecules configured for LAMP. In an embodiment, the kit further includes a second loop primer nucleic acid molecule configured for loop-mediated amplification, the second loop primer nucleic acid molecule comprising: a second target portion complementary to a second target nucleic acid sequence; and the adapter sequence. In an embodiment, the second loop primer nucleic acid molecule includes a second adapter sequence different from the adapter sequence. In an embodiment, the second target nucleic acid sequence is different than the target nucleic acid sequence. In an embodiment, the kit includes a second loop primer nucleic acid molecule complementary to a second portion of the target nucleic acid sequence, wherein the second portion of the target nucleic acid sequence is different than the target portion of the target nucleic acid sequence.

In an embodiment, the kit includes additional reagents suitable for performing LAMP. In an embodiment, the kit further includes a polymerase. In an embodiment, the kit includes dNTPs. In an embodiment, the kit includes a buffer or buffers suitable for performing LAMP with other reagents of the kit.

In an embodiment, the kit further includes additional primers suitable for performing LAMP reactions. Accordingly, in an embodiment, the kit includes a forward outer primer nucleic acid molecule complementary to an upstream portion of the target nucleic acid sequence, wherein the upstream portion is upstream of the target portion of the target nucleic acid sequence; and a backwards outer primer nucleic acid molecule complementary to a downstream portion of the target nucleic acid sequence, wherein the downstream portion is downstream of the target portion. In an embodiment, the kit further includes a forward inner primer nucleic acid molecule complementary to a second upstream portion of the target nucleic acid sequence, wherein the forward inner primer nucleic acid molecule further comprises a loop-forming portion complementary to a second downstream portion of the target nucleic acid sequence, wherein the second downstream portion is downstream of the downstream portion; and a backward inner primer complementary to a third downstream portion of the target nucleic acid sequence.

In an embodiment, the kit includes instructions for performing LAMP with the components of the kit, such as to perform one or more of the methods of the present disclosure.

In an embodiment, the kit includes reagents suitable for an internal amplification control. Accordingly, in an embodiment, the kits include internal amplification control primer nucleic acid molecules. In an embodiment, the kit includes a control targeting sequence complementary to a control portion of a control target nucleic acid sequence; and a control adapter sequence; a control displacement nucleic acid probe comprising: a control fluorophore adapter sequence; and the control adapter sequence; and a control fluorophore adapter complement nucleic acid molecule complementary to the control fluorophore adapter sequence, wherein the control fluorophore adapter sequence or the control fluorophore adapter complement nucleic acid molecule is coupled to a control fluorophore.

In an embodiment, the control fluorophore is distinguishable from the fluorophore. Accordingly, in an embodiment, the fluorophore is configured to emit fluorescence in a first wavelength range, and wherein the control fluorophore is configured to emit control fluorescence in a second wavelength range different than the first wavelength range.

### Methods of Detecting a Presence or Absence of a Target Nucleic Acid Sequence in a Sample

In another aspect, the present disclosure provides a method of detecting a presence or absence of a target nucleic acid sequence in a sample. In an embodiment, the method comprises contacting the sample with the loop primer nucleic acid molecule, the displacement nucleic acid probe, and the fluorophore adapter complement nucleic acid molecule of any of the embodiments of the present disclosure under conditions and for a time sufficient to amplify nucleic acid molecules comprising the target nucleic acid sequence; and detecting the presence or absence of fluorescence from the fluorophore.

In an embodiment, the loop primer nucleic acid molecule configured for LAMP, such as a loop primer nucleic acid molecule according to an embodiment of the present disclosure and as described elsewhere herein with respect to the kits of the present disclosure. Accordingly, in an embodiment, the loop primer nucleic acid molecule comprises a targeting sequence complementary to a target portion of a target nucleic acid sequence; and an adapter sequence.

Further, in an embodiment, the displacement nucleic acid probe according to an embodiment of the present disclosure and as discussed further herein with respect to the kits of the present disclosure. Accordingly, in an embodiment the displacement nucleic acid probe comprises a fluorophore adapter sequence; and the adapter sequence.

In an embodiment, the method includes maintaining the sample and the reagents under conditions and for a time sufficient to amplify nucleic acid molecules comprising the target nucleic acid sequence including maintaining the reagents and the sample at a temperature in a range of about 60° C. to about 70° C., such as suitable for isothermal amplification.

In an embodiment, the reagents further comprise a polymerase, such as a polymerase as described further herein.

In an embodiment, the reagents further comprise internal amplification control primer nucleic acid molecules, as described elsewhere herein. In an embodiment, the internal amplification control primer nucleic acid molecules comprise a control targeting sequence complementary to a control portion of a control target nucleic acid sequence; and a control adapter sequence; a control displacement nucleic acid probe comprising: a control fluorophore adapter sequence; and the control adapter sequence; and a control fluorophore adapter complement nucleic acid molecule complementary to the control fluorophore adapter sequence, wherein the control fluorophore adapter sequence or the control fluorophore adapter complement nucleic acid molecule is coupled to a control fluorophore. In an embodiment, the method includes maintaining the sample and the reagents under conditions and for a time sufficient to amplify nucleic acid molecules comprising the control target nucleic acid sequence; and detecting the presence or absence of control fluorescence from the control fluorophore.

In an embodiment, the fluorescence from the fluorophore and the control fluorescence from the control fluorophore are optically distinguishable, and, in this regard, are suitable to be simultaneously detected. Accordingly, in an embodiment, detecting the presence or absence of the fluorescence from the fluorophore comprises detecting fluorescence in a first wavelength range, and detecting the presence or absence of the control fluorescence from the control fluorophore comprises detecting the control fluorescence in a second wavelength range different than the first wavelength range.

In an embodiment, the reagents further comprise a second loop primer nucleic acid molecule configured for LAMP, the second loop primer nucleic acid molecule comprising: a second targeting sequence complementary to a second target portion of the target nucleic acid sequence; and the adapter sequence. In this regard, the reagents are configured to amplify and detect a second target portion of the nucleic acid sequence.

As shown in FIG. 1C, the kits of the present disclosure and methods of the present disclosure are configured to successfully amplify nucleic acid molecules including a target sequence and are suitable for fluorescence detection. In this demonstration a multiplexed LAMP amplification includes four sets of LAMP primers. Three of the primer sets (the “target” primers) are adapted to a single displacement nucleic acid probe design that uses a FAM fluorophore (solid) and they target the same piece of DNA (the “target”). While three primer sets for a single target are illustrated, it will be understood that one or more of the primer sets could react with and/or bind to different targets. For example, in an embodiment, each primer set could react with a different target. In an embodiment, two or more primer sets could react with the same target and another primer set could react with a different target. The fourth primer set is adapted to a different displacement nucleic acid probe with a Tex615 fluorophore (dashed) and it targets a different DNA strand (the “control”) when the target is added to the reaction mix the target displacement nucleic acid probe reports amplification, but the control displacement nucleic acid probe does not (N=2). When the control is added to the reaction mix the control displacement nucleic acid probe reports amplification, and the target displacement nucleic acid probe does not if there is no target in the reaction mix. While it is possible for both target and control to amplify, they compete with each other. In this regard, if target concentration is high the control may not amplify or may be very slow to do so. This demonstrates that displacement nucleic acid probes are adapter specific and universal—that multiple targets can be linked to a single probe, or parsed to different probes successfully.

### A Polypeptide

In an aspect, the present disclosure provides a polypeptide suitable for strand displacement-based nucleic acid amplification. In an embodiment, the polypeptide comprising an amino acid sequence at least 55% identical to SEQ ID NO. 25. In an embodiment, the polypeptide comprises an amino acid sequence at least 75% identical, at least 90% identical, at least 95%, at least 99% identical, or more to the amino acid sequence of SEQ ID NO. 25.

As used herein, “at least 55% identical” means that the polypeptide differs in its full length amino acid sequence by 25% or less (including any amino acid substitutions, deletions, additions, or insertions) from the polypeptide defined by, for example, SEQ ID NO: 25.

In various embodiments of any aspect of the polypeptides of the invention, the polypeptides comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO: 25.

In an embodiment, the polypeptide is a thermostable, strand-displacing polymerase. In an embodiment, the polypeptide has reverse transcriptase activity. In an embodiment, the polypeptide has manganese-inducible reverse transcriptase activity.

In an embodiment, the polypeptide includes an N-terminal affinity tag. In an embodiment, the N-terminal affinity tag is a polyhistidine tag. In an embodiment, the polypeptide includes a protease cleavage site. In an embodiment, the amino acid sequence comprises SEQ ID. NO. 26. In an embodiment, the amino acid sequence comprises an amino acid sequence according to SEQ ID NO. 27.

In another aspect, the present invention provides isolated nucleic acids encoding the polypeptide of any aspect or embodiment of the invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In a further aspect, the present invention provides nucleic acid expression vectors comprising the isolated nucleic acid of any embodiment of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In another aspect, the present invention provides recombinant host cells comprising the nucleic acid expression vectors of the invention. The host cells can be either prokaryotic or eukaryotic. The cells can be transiently or stably transfected or transduced. Such transfection and transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, cell pellet, or recovered from the culture medium. Methods to purify recombinantly expressed polypeptides are well known to the man skilled in the art.

In another aspect, the present disclosure provides a method of amplifying a nucleic acid molecule, the method comprising: contacting the nucleic acid molecule with a polypeptide according to any embodiment of the present disclosure under conditions and for a time sufficient to amplify the nucleic acid molecule.

As shown in FIG. 1, the polypeptides and methods according to the present disclosure are suitable to amplify target nucleic acid molecules, such as by LAMP. In the illustrated embodiment, 2*10⁴ copies of a synthetic RNA were detected in a 20 μL reaction using EvaGreen intercalating dye. This data also serves as a demonstration of strand displacement.

### Systems

In another aspect, the present disclosure provides systems for detection of amplification of a nucleic acid molecule in a sample. In an embodiment, the system comprises a thermal subsystem for heating a sample disposed therein, and an optical subsystem for optically excited the sample and detecting light emitted from the sample.

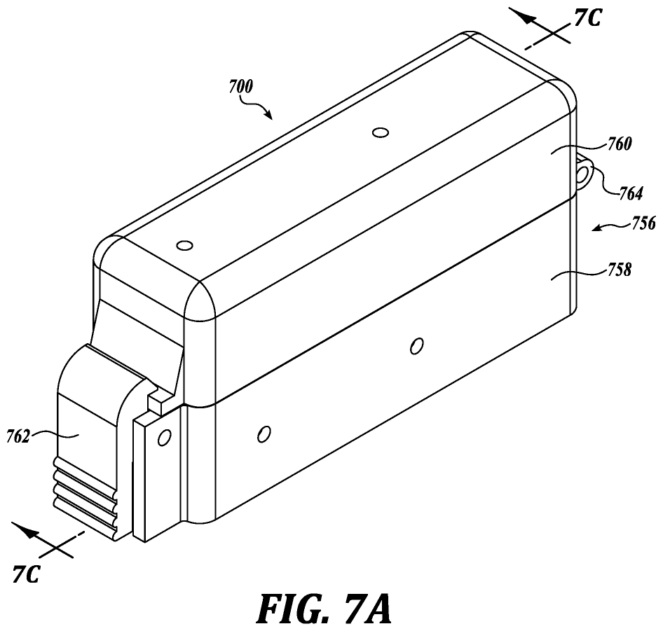

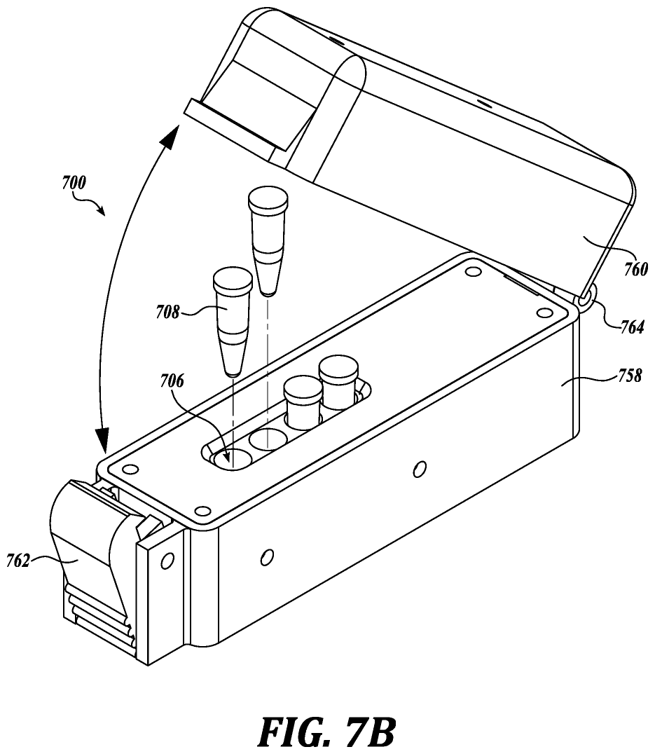

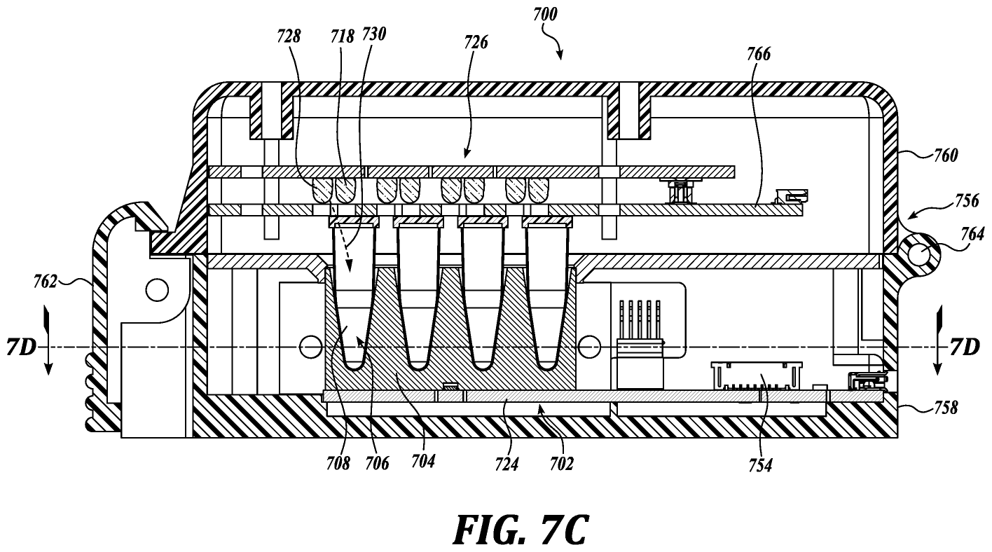

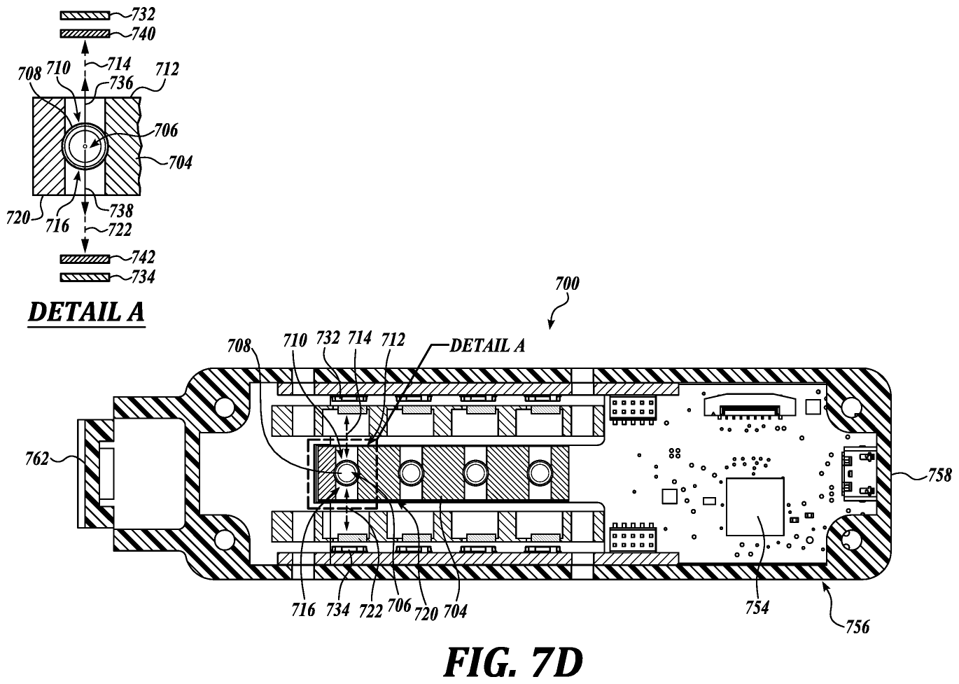

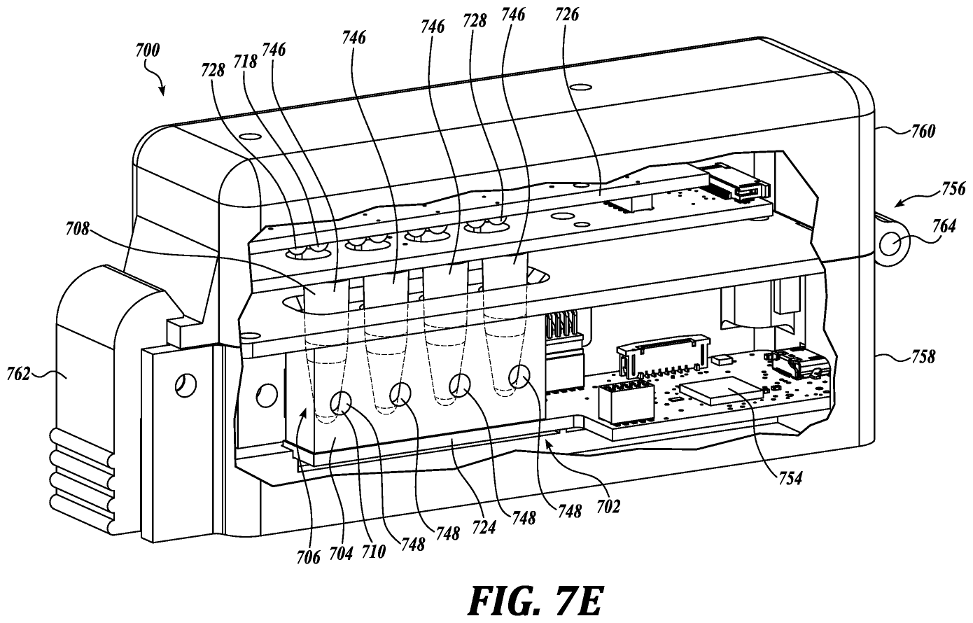

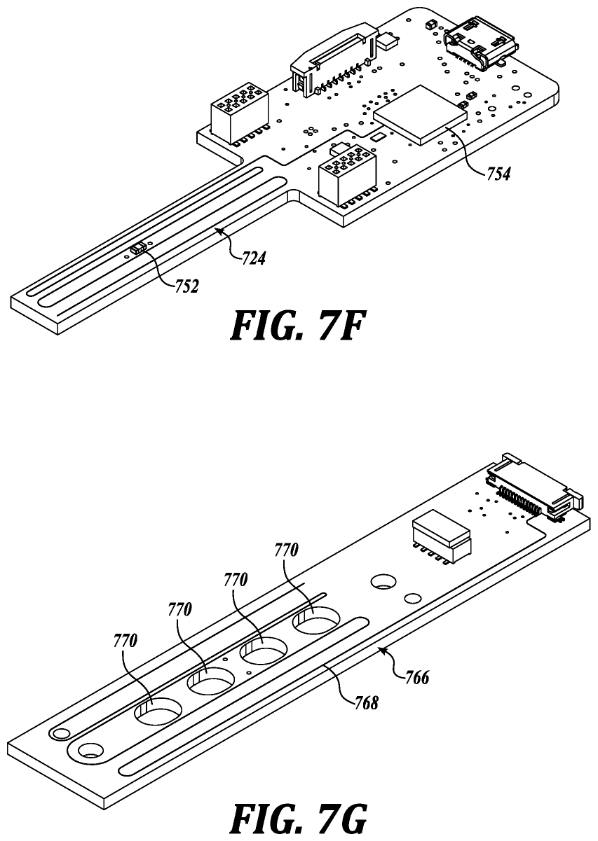

In this regard, attention is directed to FIGS. 7A-7F, in which a system 700, in accordance with an embodiment of the disclosure, is illustrated. FIG. 7A is an isometric view of the system 700. FIG. 7B is another isometric view of the system 700. FIG. 7C is a view in cross-section of the system 700. FIG. 7D is another view in cross-section of the system 700. FIG. 7E is an isometric view of the system 700. FIG. 7F is an isometric view of a heat source 724 of the system 700. FIG. 7G is an isometric view of an optical subsystem controller 766 of the system 700.

In the illustrated embodiment, the system 700 is shown to include an optical subsystem 726 for optically exciting a sample disposed in the system 700 and detecting light emitted from the sample, such as fluorescent light emitted from the sample in response to the optical excitation. The system 700 is also shown to include a thermal subsystem 702 for heating the sample, such as for a time and at a temperature suitable to amplify a nucleic acid molecule in the sample.

As shown, the thermal subsystem 702 includes a thermally conductive heat block 704 and a heat source 724 thermally coupled to the heat block 704, suitable to hear the heat block 704. In the illustrated embodiment, the heat block 704 defines a chamber 706 shaped to carry a sample holder 708 configured to carry the sample, such as an Eppendorf tube. The heat block 704 is shown to further define apertures 710 and 716 shaped to emit light from within the chamber 706, such as light from the sample in the sample holder 708 carried by the chamber 706. In this regard, the heat block 704 is shown to define a first aperture 710 disposed on a first side 712 of the heat block 704 and positioned to emit first signal light 714 from within the sample holder 708 disposed in the chamber 706; and a second aperture 716 disposed on a second side 720 of the heat block 704 opposite the first side 712 and positioned to emit second signal light 722 from within the sample holder 708 disposed in the chamber 706.

In an embodiment, the chamber 706 is shaped to carry a plurality of sample holders 746 simultaneously, and, in this regard, the system 700 is suitable to assay a plurality of samples, such as by heating a plurality of samples and optically interrogating those samples. In this regard, as shown in, for example, FIGS. 7D and 7E, the chamber 706 defines a plurality of first apertures 748 disposed on the first side 712 of the heat block 704 and positioned to emit first signal light 714 from within the plurality of sample holders 746; and a plurality of second apertures disposed on the second side 720 of the heat block 704 and positioned to emit second signal light 722 from within the plurality of sample holders 746.

As above, the system 700 includes a heat source 724 thermally coupled to the heat block 704. As shown in the FIG. 7F, the system 700 can include a heat source 724, such as an electrically resistive heat source 724 operatively coupled to a controller 754 and a power source for providing electrical power thereto.

In an embodiment, the system 700 includes a temperature sensor 752 configured to generate a temperature signal based on a temperature of the heat block 704. In the illustrated embodiment of FIG. 7F, the temperature sensor 752 is shown disposed adjacent to the heat source 724 and positioned to be in thermal and physical contact with the heat block 704.

In an embodiment, the system 700 is configured to heat the sample according to a temperature measured by the temperature sensor 752 and, in this regard, the system 700 is configured to heat the sample in the chamber 706 to a temperature suitable, for example, for nucleic acid amplification. In this regard, in an embodiment, the system 700 includes a controller 754 operatively coupled to the thermal subsystem 702, the controller 754 including logic that, when executed by the controller 754, causes the system 700 to perform operations for performing one or more methods according to the present disclosure. In an embodiment, the controller 754 includes logic that, when executed by the controller 754, causes the system 700 to perform operations including heating the heat block 704 with the heat source 724. In an embodiment, the controller 754 further includes logic that, when executed by the controller 754, causes the system 700 to perform operations including adjusting heating the heat block 704 based on the temperature signal generated by the temperature sensor 752.

As above, the system 700 of the present disclosure includes an optical subsystem 726. In an embodiment, and as shown, in FIGS. 7A-7F, the optical subsystem 726 can include a light source 728 configured to emit excitation light 730 into the chamber 706 and one or more photodetectors. In this regard, the illustrated system 700 is shown to include a first photodetector 732 positioned to receive the first signal light 714 from within the sample holder 708 through the first aperture 710; and a second photodetector 734 positioned to receive the second signal light 722 from within the sample holder 708 through the second aperture 716.

In an embodiment, the optical subsystem 726 is configured to detect light emitted from the sample in a number of different wavelength ranges. Accordingly, in an embodiment, the optical subsystem 726 includes a second light source 718 positioned to emit second excitation light into the chamber 706, wherein the excitation light 730 is in a first excitation wavelength range and the second excitation light is in a second excitation wavelength range different than the first excitation wavelength range.

Likewise, in an embodiment, the system 700 includes optical filters 740 and 742 positioned between the chamber 706 and the photodetectors 732 and 734 to optically filter light from sample or signal light emitted from the sample, to provide signals based on only a subset of the sample or signal light. In this regard, the system 700 is shown to include a first optical filter 740 positioned between the first aperture 710 and the first photodetector 732, and wherein the first optical filter 740 is configured to optically filter the first signal light 714. Additionally, in the illustrated embodiment the optical subsystem 726 further comprises a second optical filter 742 positioned between the second aperture 716 and the second photodetector 734, and wherein the second optical filter 742 is configured to optically filter the second signal light 722. In an embodiment, the second optical filter 742 is configured to optically filter a second signal light wavelength range from the second signal light 722, wherein the second optical filter 742 is configured to optically filter a second signal wavelength range from the second signal light 722, and wherein the second signal light wavelength range is different than the first signal light wavelength range.

As above, the light source 728 is positioned to illuminate a sample disposed in a sample holder 708 carried by the chamber 706. In an embodiment, the light source 728 is positioned to emit excitation light 730 and the apertures are positioned to emit sample or signal light such that excitation light 730 is not generally received by the photodetector(s). In this regard, in an embodiment, the light source 728 is positioned to emit the excitation light 730 in a direction orthogonal to a major axis 736 of the first aperture 710 and major axis 738 of the second aperture 716. While the light source 728 is shown positioned at a top side of the system 700, it will be understood that the light source 728 can be disposed in other position about the system 700, such as on a bottom side or another side of the system 700.

In the illustrated embodiment, the system 700 is shown to include an optical subsystem controller 766 suitable to control or modify operation of the optical subsystem 726, such an in conjunction with the controller 754. As shown in FIG. 7G, the optical subsystem controller 766 is configured to receive power, such as electrical power from a power source, as well as a plurality of optical apertures 770, positioned to allow excitation light 730 from the plurality of light source 728 to pass through to sample holders carried by the heat block 704. In the illustrated embodiment, the optical subsystem controller 766 is also shown to include a second heat source 768, such as a second electrically resistive heat source positioned to heat side of the heat block 704 opposite the first heat source 724.

In an embodiment, the controller 754 includes logic for operating the light source(s) to excite sample(s) disposed in the system 700 and generate signals based on light emitted from the sample(s). In this regard, in an embodiment, the controller 754 is operatively coupled to the optical subsystem 726, the controller 754 including logic that, when executed by the controller 754, causes the system 700 to perform operations including emitting the excitation light 730 with the light source 728; generating a first sample signal with the first photodetector 732 based on the first signal light 714 received by the first photodetector 732; and generating a second sample signal with the second photodetector 734 based on the second signal light 722 received by the second photodetector 734. In an embodiment, the controller 754 includes logic that, when executed by the controller 754, causes the system 700 to perform operations including emitting the second excitation light with the second light source 718; generating a second sample signal with the second photodetector 734 based on the second signal light received by the second photodetector 734.

As shown, the system 700 further includes a housing 756 shaped to carry the optical subsystem 726 and the thermal subsystem 702. In an embodiment, the housing 756 is suitable to protect the optical subsystem 726 and the thermal subsystem 702, as well as shield the optical subsystem 726 from stray light, which may interfere with signal detection.

As shown, the housing 756 comprises a case 758 shaped to carry the heat block 704; and a lid 760 hingedly coupled to the case 758, such as through hinge 764. Accordingly, in an embodiment, the housing 756 is configured to shield the optical subsystem 726 from light outside of the housing 756 when the lid 760 is in the closed configuration. In an embodiment, the housing 756 is configured to encase the optical subsystem 726 and the thermal subsystem 702 when the lid 760 is in a closed configuration. See, for example, FIGS. 7A and 7C. Further, in an embodiment, the housing 756 is configured to allow access to the chamber 706 in introducing sample holder 708 to the heat block 704 in an open configuration. See, for example, FIG. 7B. As shown, the case 758 includes a hinge releasably coupling the lid 760 to the case 758. Additionally, in an embodiment, the housing 756 includes a latch 762 configured to selectively and releasably close the lid 760 to the case 758, as shown.

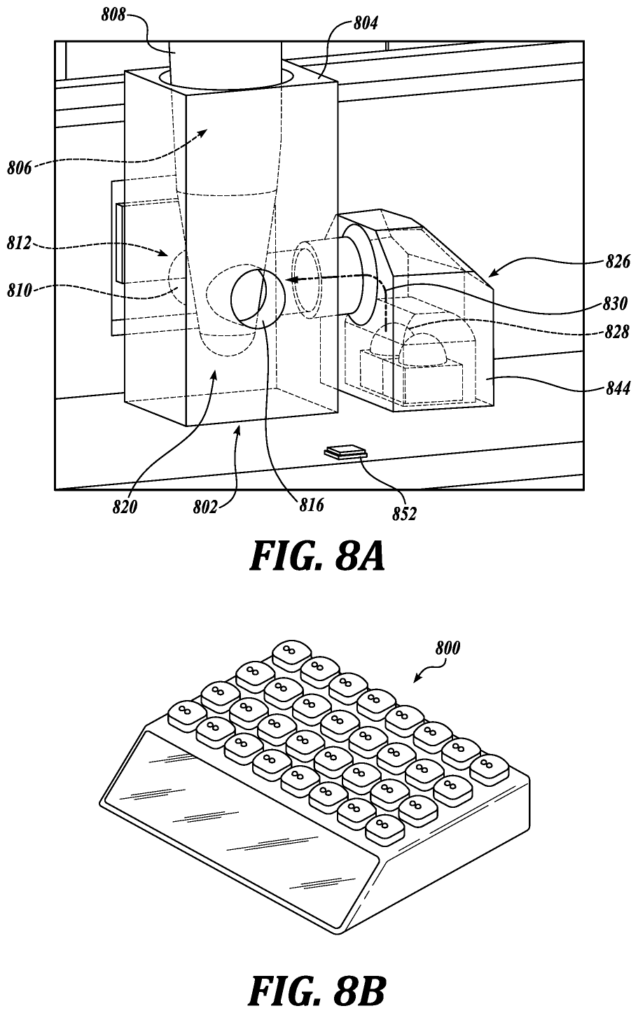

In an embodiment, the systems of the present disclosure include an optical waveguide configured to guide the excitation light from the light source into the chamber. In that regard, attention is directed to FIGS. 8A and 8B in which a system 800, according to an embodiment of the present disclosure is illustrated. FIG. 8A is a partial isometric view of the system 800. FIG. 8B is another isometric view of the system 800.

In an embodiment, the system 800 is an embodiment of the system 700, discussed further herein with respect to FIGS. 7A-7F. Accordingly, in an embodiment, the system 800 includes a thermal subsystem 802 comprising: a thermally conductive heat block 804 defining a chamber 806 shaped to carry a sample holder 808 configured to carry the sample; a first aperture 810 disposed on a first side 812 of the heat block 804 and positioned to emit first signal light from within the sample holder 808 disposed in the chamber 806; and a second aperture 816 disposed on a second side 820 of the heat block 804 opposite the first side 812 and positioned to emit second signal light from within the sample holder 808 disposed in the chamber 806; and a heat source thermally coupled to the heat block 804; and an optical subsystem 826 comprising: a light source 828 configured to emit excitation light into the chamber 806; a first photodetector positioned to receive the first signal light from within the sample holder 808 through the first aperture 810; and a second photodetector positioned to receive the second signal light from within the sample holder 808 through the second aperture 816.

As shown, the system 800 includes an optical waveguide 844 configured to guide the excitation light from the light source 828 into the chamber 806. The optical waveguide 844 is configured to direct the excitation light from the light source 828 to the chamber 806 more efficiently than simply through the air and, in this regard, may be suitable to provide more light to a sample and, thereby, increase signal intensity. In the illustrated embodiment, the system 800 includes multiple light sources, including the light source 828, optically coupled to the optical waveguide 844. In this regard, the optical waveguide 844 is configured to guide, for example, excitation light having multiple wavelength ranges to the chamber 806.

While not illustrated, in an embodiment, the system 800 includes an optical waveguide configured to guide signal light to the photodetector.

In the illustrated embodiment, the heat block 804 is configured to carry a single sample holder 808. See FIG. 8A. In an embodiment, the heat block 804 is a first heat block 804, wherein the system 800 further comprises a second thermally conductive heat block defining a second chamber shaped to carry a second sample holder; third aperture disposed on a first side of the second heat block and positioned to emit third signal light from within the second sample holder; and a fourth aperture disposed on a second side of the second heat block opposite the first side of the second heat block and positioned to emit fourth signal light from within the second sample holder. (Not shown). In this regard, the heat block 804 shown in FIG. 8A is repeatedly provided along with corresponding light sources, optical waveguides, etc. Accordingly, the system 800 is provided with a plurality of heat blocks configured to individually carry, heat, and interrogate a plurality of sample holders, such as within the heat blocks.

As shown, the system 800 is shown to include a temperature sensor 852 adjacent to and in thermal communication with the heat block 804. As discussed further herein with respect to FIGS. 7A-7F, the temperature sensor 852 is suitable to monitor and adjust heating of the heat block 804. In an embodiment, the system 800 includes a plurality of temperature sensors 852 thermally coupled to or otherwise in thermal communication with the plurality of heat blocks 804.

### Methods for Detecting Amplification of a Nucleic Acid Molecule in a Sample

In another aspect, the present disclosure provides methods for detecting amplification of a nucleic acid molecule in a sample. In an embodiment, the methods can include the use of one or more systems in accordance with the present disclosure, such as those describe further herein with respect to FIGS. 7A-7F and FIGS. 8A and 8B.

In an embodiment, the method comprises heating a thermally conductive heat block with a heat source thermally coupled to the heat block, wherein the heat block defines a chamber shaped to carry a sample holder carrying the sample. In an embodiment, the heat block is a heat block as discussed further herein with respect to FIGS. 7A-7F and/or FIGS. 8A and 8B. In an embodiment, the sample is a sample containing or suspected of containing a target nucleic acid molecule. In an embodiment, the sample contains reagents suitable for amplifying and detecting amplification of the nucleic acid molecule in the sample. In an embodiment, heating the thermally conductive heat block with the heat source is configured to heat the sample to a temperature, or series of temperatures, for a time sufficient to amplify the nucleic acid molecule in the sample. In an embodiment, the method includes adjusting heating the heat block based on a temperature signal generated by the temperature sensor, such as to maintain a temperature of the sample within a predetermined temperature range suitable for sample amplification.

As discussed further herein, in an embodiment, the heat block defines a first aperture disposed on a first side of the heat block and positioned to emit first signal light from within the sample holder disposed in the chamber; and a second aperture disposed on a second side of the heat block opposite the first side and positioned to emit second signal light from within the sample holder disposed in the chamber.

In an embodiment, the method includes emitting, with a light source, the excitation light into the sample holder. In an embodiment, the excitation light is configured to excite a detectable agent within the sample, such as a fluorescent dye or other detectable marker configured to selectively bind to an amplicon of the nucleic acid amplification reaction. In an embodiment, the light source(s) can be turned on and off. In an embodiment and as discussed elsewhere herein, the method includes emitting excitation light from multiple light sources having, for example, multiple and different wavelength ranges.

In response to such excitation light, the sample, including the detectable agent can emit signal light, such as in the form of fluorescent or scattered light emitted from the sample. Accordingly, in an embodiment, the methods further includes generating a first sample signal with a first photodetector based on the first signal light received by the first photodetector through the first aperture; and generating a second sample signal with a second photodetector based on the second signal light received by the second photodetector through the second aperture. Such first and second sample signals can be used to determine whether one or more analytes, such as one or more amplicons, are present in the sample.

The order in which some or all of the process steps are described in each process should not be deemed limiting. Rather, one of ordinary skill in the art having the benefit of the present disclosure will understand that some of the process steps may be executed in a variety of orders not illustrated, or even in parallel.

### Non-Transitory, Machine-Readable Storage Media

In another aspect, the present disclosure provides non-transitory, machine-readable storage medium having instructions stored thereon, which when executed by a processing system, cause the processing system to perform certain operations. In an embodiment, such operations are suitable to perform one or methods according to the present disclosure and/or are suitable for use with one or more systems of the present disclosure, such as discussed further herein with respect to FIGS. 7A-7F.

Accordingly, in an embodiment, the non-transitory, machine-readable storage medium having instructions stored thereon, which when executed by a processing system, cause the processing system to perform operations including: heating a thermally conductive heat block with a heat source thermally coupled to the heat block, wherein the heat block defines a chamber shaped to carry a sample holder carrying a sample; a first aperture disposed on a first side of the heat block and positioned to emit first signal light from within the sample holder; and a second aperture disposed on a second side of the heat block opposite the first side and positioned to emit second signal light from within the sample holder; emitting, with a light source, excitation light into the sample holder disposed in the chamber; generating a first sample signal with a first photodetector based on the first signal light received by the first photodetector through the first aperture; and generating a second sample signal with a second photodetector based on the second signal light received by the 25 second photodetector through the second aperture.

Some processes explained above are described in terms of computer software and hardware. The techniques described may constitute machine-executable instructions embodied within a tangible or non-transitory machine (e.g., computer) readable storage medium, that when executed by a machine will cause the machine to perform the operations described. In an aspect, the present disclosure provides non-transitory, machine-readable storage media for performing one or more methods of the present disclosure, such as with one or more of the systems of the present disclosure. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

### EXAMPLES

### Example 1: Materials and Methods

Preparation of Tfpol Polymerase

Plasmid preparation and protein expression and purification were performed as previously described (Panpradist N, Wang Q, Ruth P S, Kotnik J H, Oreskovic A K, Miller A, Stewart S W A, Vrana J, Han P D, Beck I A, Starita L M, Frenkel L M, Lutz B R. Simpler and faster Covid-19 testing: Strategies to streamline SARS-CoV-2 molecular assays. EBioMedicine. 2021 February; 64:103236. doi: 10.1016/j.ebiom.2021.103236. Epub 2021 Feb. 12. Erratum in: EBioMedicine. 2021 April; 66:103296. PMID: 33582488; PMCID: PMC7878117).

Primer and IAC Design

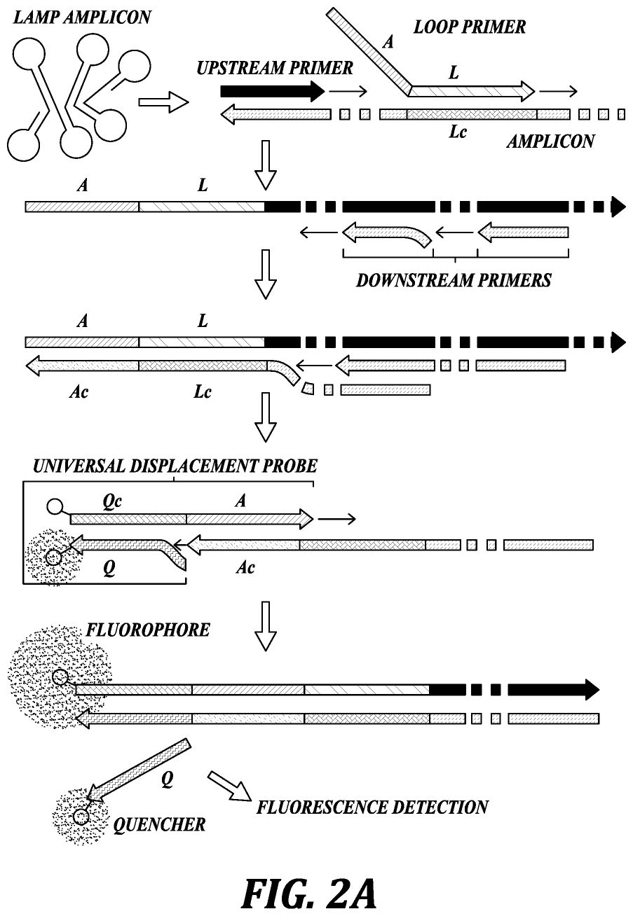

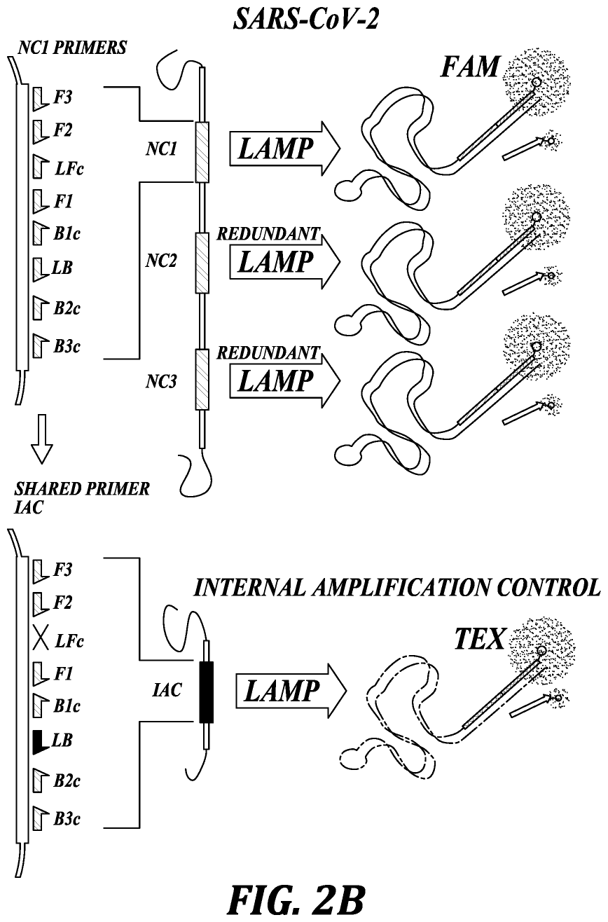

Three sets of LAMP primers (Table 1) targeting three different regions of the SARS-CoV-2 nucleocapsid phosphoprotein were designed manually using the primer design feature of Geneious 8.1.9 against the SARS-CoV-2 reference sequence (GenBank accession number: NC_045512). IDT Oligo analyzer and NUPACK were used to evaluate designs in silico. Each target design consists of the six conventional LAMP primers: F3, B3, FIP, BIP; LF, and LR. The IAC was designed using a composite primer technique for LAMP. IAC template sequence was derived from target region “NC1” by substituting the target loop primer binding sites with engineered sequences. One of the engineered IAC loop sites was used as an IAC loop primer while the other was omitted, so that the IAC assay uses a single loop primer (LFc mut in FIG. 2B). For each primer set a loop primer was modified by the addition of an engineered probe adapter sequence at its 5′ end, with all targets sharing a common adapter and the control assay using a second unique adapter sequence.

TABLE 1
Primer, Probe, and control sequences for the SARS-COV-2 mRT-LAMP.
For Primers and probes, F2/B2 sequences are underlined, non-template
linker sequence sare italicized, and adapter sequences are shown in bold.

| SARS-COV-2 NC1 | | |
|---|---|---|
| Primers | Sequence | SEQ ID NO |
| NC1 FIP | CCACTGCGTTCTCCATTCTTTTCCCCGCAT | SEQ ID NO. 1 |
| | TACGTTTGGT | |
| NC1 BIP | GCGATCAAAACAACGTCGGTTATTGCCAT | SEQ ID NO. 2 |
| | GTTGAGTGAGAGCG | |
| NC1 LF | TGGTTACTGCCAGTTGAATCT | SEQ ID NO. 3 |
| NC1 LB + Target | ACCAACACCTCACATCACACATAATAG | SEQ ID NO. 4 |
| adapter | GTTTACCCAATAATACTGCGTCTTG | |
| NC1 F3 | TGGACCCCAAAATCAGCG | SEQ ID NO. 5 |
| NC1 B3 | ATCTGGACTGCTATTGGTGTTA | SEQ ID NO. 6 |
| SARS-COV-2 NC2 | | |
| Primers | Sequence | SEQ ID NO |
| NC2 FIP | CAGCTTCTGGCCCAGTTCCTGTGGTGGTG | SEQ ID NO. 7 |
| | ACGGTAAAATG | |
| NC2 BIP | CTTCCCTATGGTGCTAACAAAGTCCAATG | SEQ ID NO. 8 |
| | TGATCTTTTGGTGTATTCA | |
| NC2 LF | GTAGTAGAAATACCATCTTGGACT | SEQ ID NO. 9 |
| NC2 LB | ACCAACACCTCACATCACACATAATAA | SEQ ID NO. 10 |
| + Target | TATGGGTTGCAACTGAGGGAG | |
| adapter | | |
| NC2 F3 | CTACTACCGAAGAGCTACCAG | SEQ ID NO. 11 |
| NC2 B3 | GCAGCATTGTTAGCAGGATTG | SEQ ID NO. 12 |
| SARS-COV-2 NC3 | | |
| Primers | Sequence | SEQ ID NO |
| NC3 FIP | TGTGTAGGTCAACCACGTTCTGCTTCAGC | SEQ ID NO. 13 |
| | GTTCTTCGGA | |
| NC3 BIP | GTGCCATCAAATTGGATGACAAAGGTTTT | SEQ ID NO. 14 |
| | GTATGCGTCAATATGCTTATTCAG | |
| NC3 LF + Target | ACCAACACCTCACATCACACATAATAT | SEQ ID NO. 15 |
| adapter | CCATGCCAATGCGCGACA | |
| NC3 LB | CCAAATTTCAAAGATCAAGTCAT | SEQ ID NO. 16 |
| NC3 F3 | GACCAGGAACTAATCAGACAAG | SEQ ID NO. 17 |
| NC3 B3 | GCTTGAGTTTCATCAGCCTTC | SEQ ID NO. 18 |
| IAC (NC1) | | |
| primer | Sequence | SEQ ID NO |
| IAC FL + Control | ACCACACCTACCACCACTAATAACTAA | SEQ ID NO. 19 |
| adapter | CTCCAGCCATCCTCACCATC | |
| SARS-COV-2 | | |
| UDP | Sequence | SEQ ID NO |
| Target (CoV) UDP | FITC- | SEQ ID NO. 20 |
| Probe | CCATCAGCACCAAGACTACCCACCTCGC | |
| | CACCAAACCAACACCTCACATCACACA | |
| | TAATA | |
| Target (CoV) UDP | TTGGTGGCGAGGTGGGTAGTCTTGGTGCT | SEQ ID NO. 21 |
| Quencher | GATGG-Iowa Black® FQ | |
| IAC UDP | Sequence | SEQ ID NO |
| Control (IAC) UDP | Tex615- | SEQ ID NO. 22 |
| Probe | CCTGACCACTTCCGAACCCAACCACCTAC | |
| | GACAGACCACACCTACCACCACTAATA | |
| | ACTAA | |
| Control (IAC) UDP | CTGTCGTAGGTGGTTGGGTTCGGAAGTG | SEQ ID NO. 23 |
| Quencher | GTCAGG - BHQ®-2 | |
| IAC Template | Sequence | SEQ ID NO |
| IAC SSDNA | AAT GGA CCC CAA AAT CAG CGA AAT | SEQ ID NO. 24 |
| | GCA CCC CGC ATT ACG TTT GGT GGA | |
| | CCC TCT GGA GTC AAT GGG TGG TGC | |
| | CAG AAT GGA GAA CGC AGT GGG GCG | |
| | CGA TCA AAA CAA CGT CGG CCC CAA | |
| | GTT GAT CTC CAG CCA TCC TCA CCA TCG | |
| | TTC ACC GCT CTC ACT CAA CAT GGC | |
| | AAG AAT TAA CAC CAA TAG CAG TCC | |
| | AGA TG | |

Universal Displacement Probe Design

Two engineered universal displacement probes (UDP) corresponding to the target adapter or IAC adapter sequence were designed. Each UDP consists of an oligonucleotide duplex with a 3′ overhang and a fluorophore quencher pair the adapter sequence is located at the 3′ overhang position, with a fluorophore spacer sequence at the 5′ end and a 5′ terminal fluorophore (6-FAM or TEX615). The quencher (Iowa Black® FQ or Black hole Quencher®-2) sequence is complementary fluorophore spacer sequence and is labeled with a 3′ dark quencher so that it quenches the fluorophore when annealed. Probe adapters and universal displacement probe sequences were generated from randomized sequence and manually modified in Geneious, using Oligo analyzer and NUPACK as a secondary analysis tools, to minimize dimer and hairpin structures within and between the probes and adapted loop primers. All designs were tested individually and multiplexed in combination against synthetic dsDNA target (IDT gBlock) and ssDNA IAC (IDT Ultramer) fragments to inform iterative design changes to individual assays. Final design iterations are reported.

Patient Samples

A panel of 102 human respiratory specimens was used to evaluate our mLAMP assay performance. These specimens collected from nasal or nasopharyngeal swabs were suspended in 3 mL viral transport medium (Becton Dickinson 220220), aliquoted, and stored at −80° C. until testing as described (Panpradist N, Wang Q, Ruth P S, Kotnik J H, Oreskovic A K, Miller A, Stewart S W A, Vrana J, Han P D, Beck I A, Starita L M, Frenkel L M, Lutz B R. Simpler and faster Covid-19 testing: Strategies to streamline SARS-CoV-2 molecular assays. EBioMedicine. 2021 February; 64: 103236. doi: 10.1016/j.ebiom.2021.103236. Epub 2021 Feb. 12. Erratum in: EBioMedicine. 2021 April; 66:103296. PMID: 33582488; PMCID: PMC7878117). The panel was originally characterized by OpenArray (ThermoFisher Scientific) to contain at least 30 COVID-POS across a wide range of concentrations and 30 COVID-negative samples as well as other samples identified as positive for other respiratory diseases including Influenza, seasonal Coronavirus, Adenovirus, and Enterovirus. Samples were reassessed in house for the presence of SARS-CoV-2 RNA, as described below, to account for losses during freeze-thaws, storage, or extraction. In-house results were used as the reference standard. Specimens were collected and tested for SARS-CoV-2 infection as part of the Seattle Flu Study, as approved by the Institutional Review Board at the University of Washington (IRB #: STUDY0006181). Informed consent was obtained for all participant samples, including for use of de-identified, remnant specimens.

Patient Sample Preparation

Specimens were extracted using the QIAamp Viral RNA Mini Kit (Qiagen #52906) according to the manufacturer's protocol. 100 μL of sample was mixed with 40 μL negative VTM (to reach the manufacturer's recommended 140 μL input), extracted, and eluted in 70 μL buffer. 5 μL aliquots were prepared for single use to avoid free thawing and stored at −80° C. until use.

mRTLAMP Protocol

20 μL mRT-LAMP reaction contains 5 mM DTT, 8 mM magnesium sulfate, 20 mM Tris-HCl, 10 mM ammonium sulfate, 10 mM KCl, 0.5% (v/v) Triton X-100, 1 μM of each FIP and BIP primers, 500 nM of each LF and FB primers, 200 nM of each FV and BV primers, 200 nM FAM-tagged UDP probe and TEX 615 UDP probe, 300 nM Quencher 1 and Quencher 2 probes, 10 units of RNasin® Plus Ribonuclease Inhibitor (Promega, N2611), 6 units of WarmStart® RTx (NEB, M0380L), 0.7 μg TFpol polymerase, and 2 units of thermostable inorganic pyrophosphatase (NEB, M0296L). 5 μL of extracted RNA was added to 15 μL mLAMP reaction mixture and incubated at 63.3° C. for 1 hour. Fluorescence measurements for FAM and TEX 615 signal, indicating SARS-CoV-2 and IAC amplification, respectively, were taken every 25 seconds (accounting for 13 second cycle and read times).

RT-PCR Protocol

The RT-PCR protocol was prepared as previously described (Panpradist N, Wang Q, Ruth P S, Kotnik J H, Oreskovic A K, Miller A, Stewart S W A, Vrana J, Han P D, Beck I A, Starita L M, Frenkel L M, Lutz B R. Simpler and faster Covid-19 testing: Strategies to streamline SARS-CoV-2 molecular assays. EBioMedicine. 2021 February; 64:103236. doi: 10.1016/j.ebiom.2021.103236. Epub 2021 Feb. 12. Erratum in: EBioMedicine. 2021 April; 66:103296. PMID: 33582488; PMCID: PMC7878117). Each 20 μL RT-PCR reaction contains 5 mM DTT, 200 μM ea. dNTP, 1× of either N1, N2, or RP primer/probe mix (IDT, 10006770), 80 mM Tris-sulfate, 20 mM ammonium sulfate, 4 mM magnesium sulfate, 5% (v/v) glycerol, 5% (v/v) DMSO, 0.06% (v/v) IGEPAL CA-630, 8.4% (w/v) trehalose, 0.05% (v/v) Tween-20, 0.5% (v/v) Triton X-100, 7.5 U reverse transcriptase (NEB M0380L), and 2.5 U polymerase (NEB M0481L). 5 μL of extracted RNA was added to the 15 μL RT-PCR reaction mixture and subjected to 5 minutes at 55° C., 1 minutes of 94° C. and 45 cycles of 1 second 94° C. and 30 seconds at 57° C. and read using FAM channel on a CFX96 (Biorad). Each clinical sample was run with one technical replicate for each N1, N2, or RP assay, along with standards using synthetic RNA templates prepared in-house and quantified using ddPCR as described (Panpradist N, Wang Q, Ruth P S, Kotnik J H, Oreskovic A K, Miller A, Stewart S W A, Vrana J, Han P D, Beck I A, Starita L M, Frenkel L M, Lutz B R. Simpler and faster Covid-19 testing: Strategies to streamline SARS-CoV-2 molecular assays. EBioMedicine. 2021 February; 64: 103236. doi: 10.1016/j.ebiom.2021.103236. Epub 2021 Feb. 12. Erratum in: EBioMedicine. 2021 April; 66:103296. PMID: 33582488; PMCID: PMC7878117). Cq and SQ values were exported from Bio-Rad CFX Maestro 1.1 software (version 4.1.2433.1219) using the RFU threshold of 50 across all datasets.

Sequence Analysis

Genomic sequences of SARS-COV-2 were downloaded from GISAID.org. Criteria for inclusion were: complete, high coverage sequences with an identified lineage collected between (date1) and (date2) submitted prior to (date of download). This sequence library was screened for identity matches with the primer binding regions of the NC1, NC2, NC3 assays using the packages Biostrings and seqnir for R.

### Example 2: Results

To efficiently combine mRT-LAMP assays and differentiate between target and IAC amplification in a crude sample matrix requires two key features: a target specific probe technology (FIG. 2) and a strand displacement polymerase with low non-template amplification. We developed fluorescent universal displacement probes (UDPs) to allow multiplexed assays to be combined or parsed into fluorescence channels with a minimum number of probes. UDPs themselves are engineered sequences that use a universal adapter sequence on a loop primer for target-specific detection (FIG. 2A). In the configuration presented here, three independent SARS-CoV-2 targets are designed to report to a single green (6-FAM) fluorescent probe, and the IAC is designed to report to a red (TEX615) fluorescence channel (FIG. 2B). We previously developed an in-house thermostable strand displacement polymerase (TFpol) with very low nonspecific amplification that is amenable to multiplexing. The TFpol design was similar to the chimeric polymerase method of Morant using the poll polymerase of Thermus Thermophilus as the backbone, an enzyme shown to be tolerant of many polymerase inhibitors. UDPs and TFpol combine to allow for a flexible and robust mLAMP system, compatible with multiple target redundancy, IAC controls, and potential for reduced sample preparation.

Analytical Performance of SARS-Cov-2 mRT-Lamp

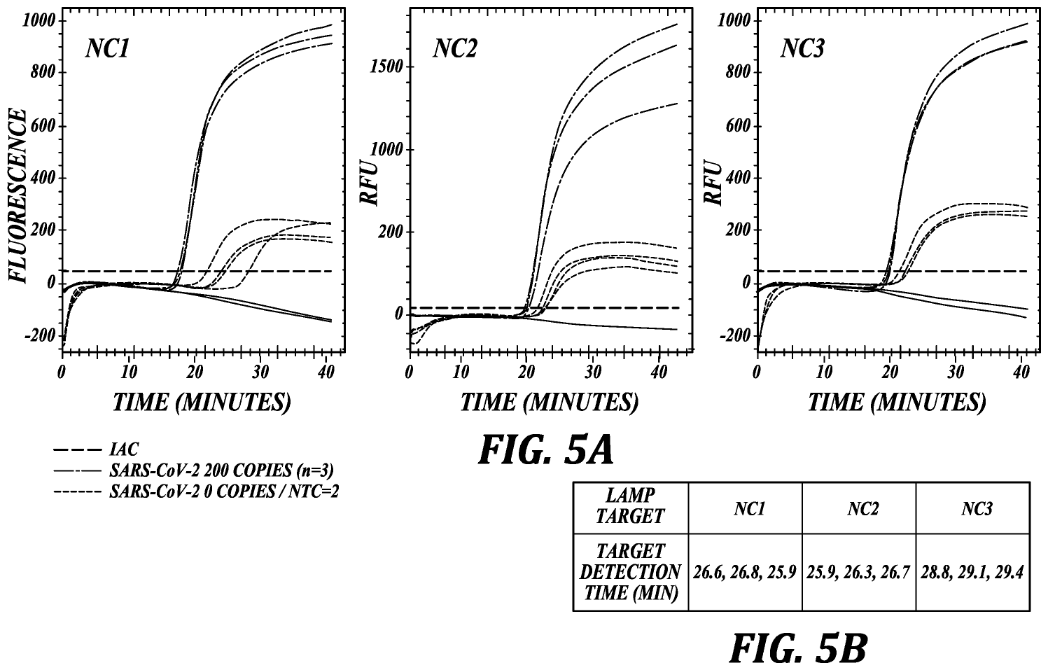

Functionality of the individual redundant targets in the mRT-LAMP was verified using synthetic RNA fragments corresponding to NC1, NC2, or NC3 mRT-LAMP assay footprints. All three target regions generated detectable amplification (FIG. 5A) with similar average reaction times with 200 copies of transcript RNA (NC1: 26.4 min, NC2: 26.3, NC3: 28.7 min; FIG. 5B).

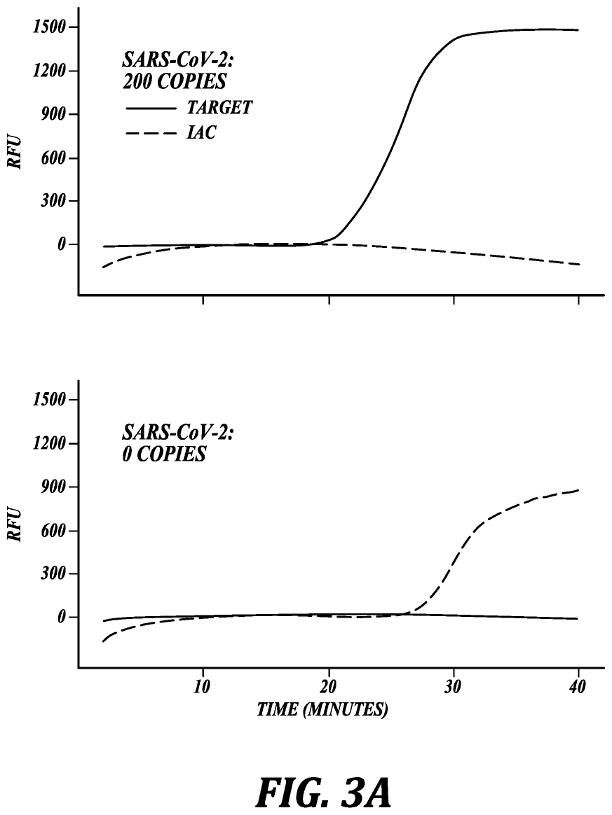

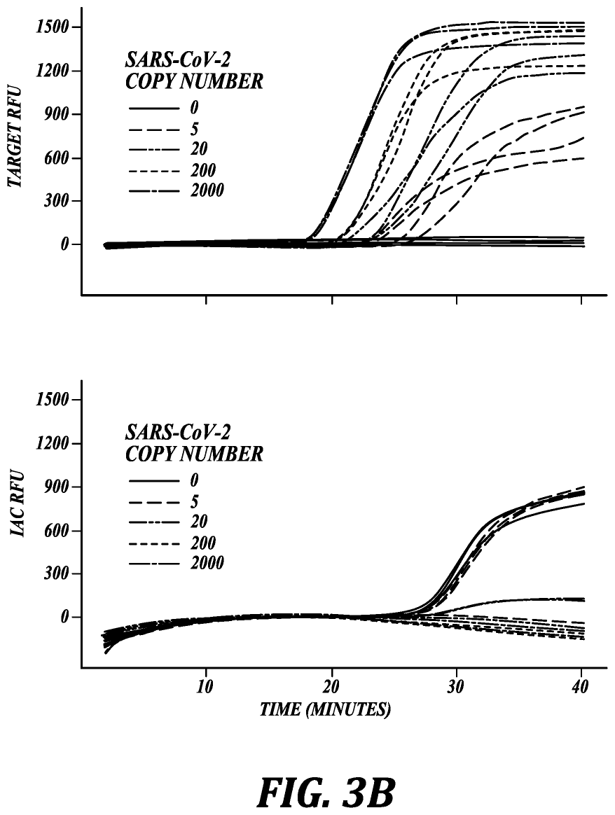

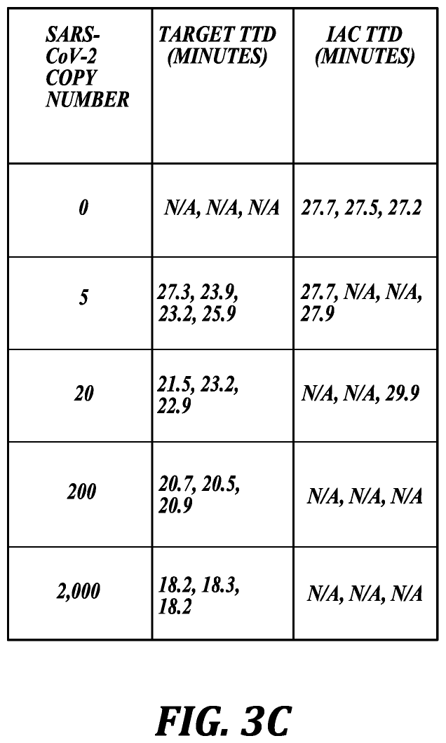

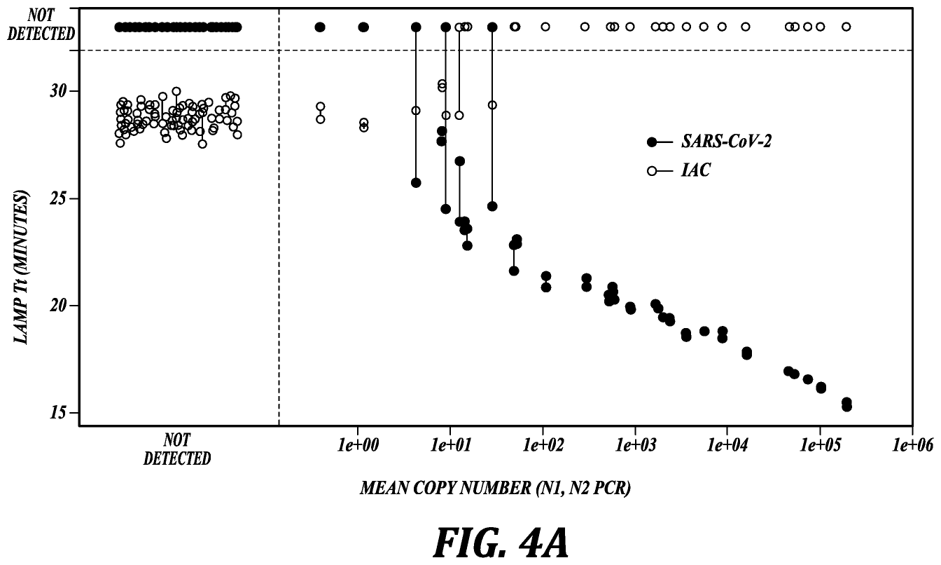

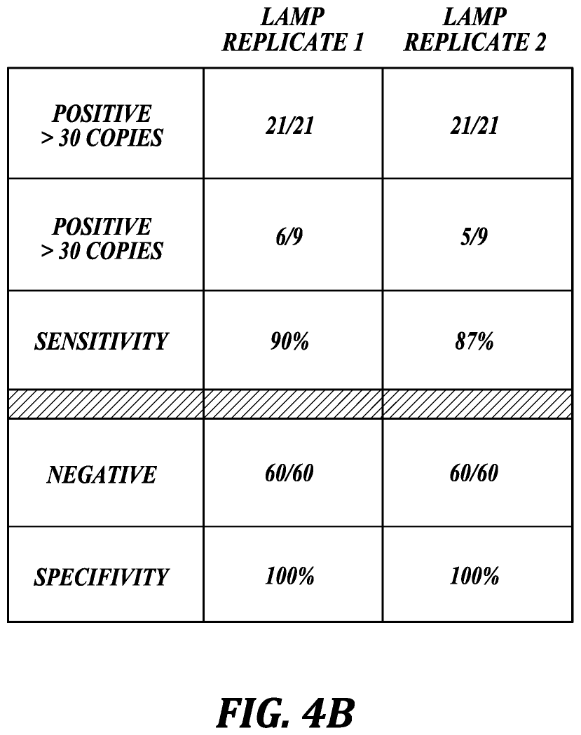

The multiplexed assay was evaluated with synthetic target RNA containing all three target regions in the presence of 105 copies of a single-stranded DNA internal amplification control (FIG. 3A). The amount of IAC was chosen to allow detection of low-copy targets prior to detection of the IAC, in order to reduce resource competition between target and control amplifications. This timing differential is possible because of the reduced rate of amplification with a single loop primer in the IAC primer set, when compared to the target assays with a standard complement of LAMP primers. Input of 200 SARS-CoV-2 RNA copies (FIG. 3A, top) resulted in detection of green fluorescence in about 21 minutes, while the IAC was not detected. For zero SARS-CoV-2 input copies, there was no target amplification, and the IAC signal was detected by red fluorescence at about 27.5 minutes (FIG. 3A, bottom). This behavior is ideal for a shared-primer IAC strategy, permitting detection of the target organism or, alternatively, validating the assay chemistry with the control reaction in the absence of target NAs. The analytical sensitivity was assessed with synthetic RNA target (FIG. 3B). All reactions containing target RNA were positive for target amplification and all NTC reactions detected IAC amplification and were negative for target detection (FIG. 3A). Some IAC amplifications were detected in low copy reactions containing target RNA (FIG. 3A), but their presence did not compromise target detection. The assay detected down to 5 copies per reaction (n=4), and all reactions had threshold times of 30 minutes or less for both the target and IAC (FIG. 3C).

Tolerance to Transport Media

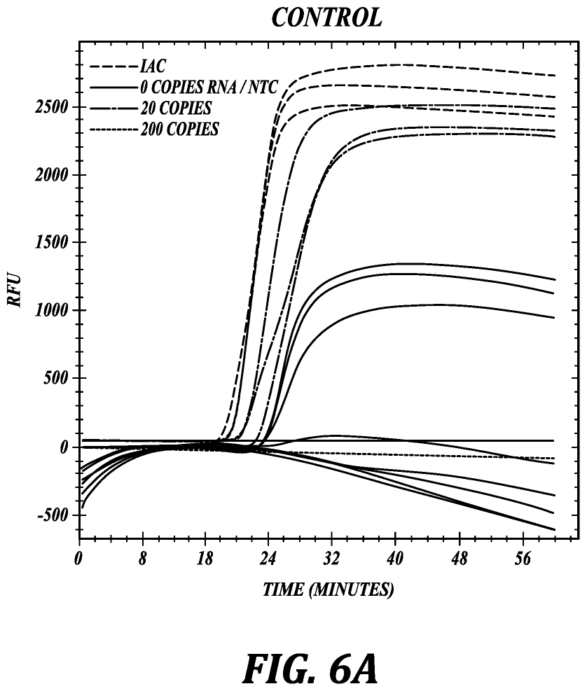

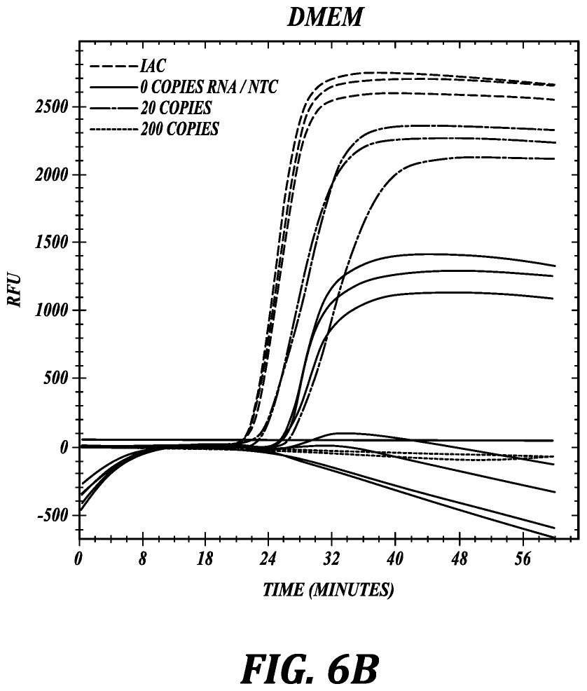

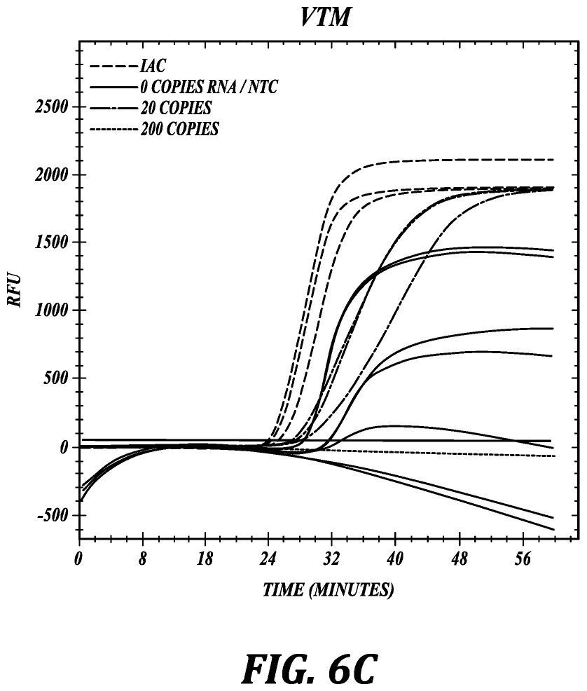

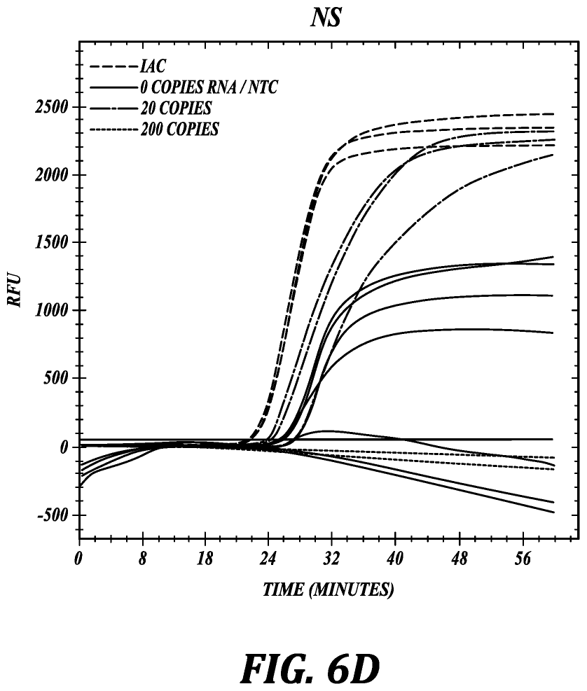

To evaluate the tolerance of the assay to potential media contaminants, a selection of commercially available co buffered transport reagents were spiked into reactions with a 25% final concentration. For a 20 μL total reaction volume, 5 μL of 1×DMEM (11965-06, Gibco), 1×VTM (BD 220527, Copan), 1×PBS (SH30256.01, GE) or 0.9% sodium chloride (diluted from 5M stock 71386-1L, Sigma) was added into the mRT-LAMP reactions with final synthetic SARS-CoV-2 RNA of 0, 20, or 200 copies (FIG. 6). Successful target amplification was observed for all samples containing template under all buffer conditions.

Performance with Extracted Clinical Specimens:

The SARS-CoV-2 mRT-LAMP was evaluated against a collection of pre-extracted patient specimens. Of the 102 samples evaluated by RT-PCR, 93 were determined to contain human origin material by positive RNase P (RP) results; all samples that were negative for RP were also negative for SARS-CoV-2. Of the 93 specimens verified to contain human material, 60 were found to be negative for SARS-CoV-2, and 30 were found to be positive by both reference RT-RCR assays. The three remaining samples were positive for SARS-CoV-2 by one reference RT-PCR assay and negative by the second, resulting in an inconclusive classification. All samples that were negative for RP or inconclusive for SARS-CoV-2 by RT-PCR were excluded from analysis. Clinical samples were run in duplicate mRT-LAMP reactions.

The mRT-LAMP was able to detect negatives with 100% specificity in both sets of replicates, with detection of the IAC but no target signal (FIG. 3). Conversely, sensitivity for the two replicates was 90% (27/30) and 87% (26/30), respectively. For samples found to have more than 30 copies/mRT-LAMP reaction by reference RT-PCR, sensitivity was improved to 100% (21/21) for both replicates. The OpenArray characterization of the verified samples found 28/90 contained other respiratory infections; all were correctly called (1 positive (coinfection), 27 negative).

### Example 3: Discussion

This initial validation of a multiplexed reverse transcription LAMP assay is a further step towards more resilient point-of-care NAAT technologies with convenient implementation and development. The assay supports robust but basic functionality with competitive sensitivity, speed, and a low complexity fluorescence detection system. To support our efforts, we utilized our in-house chimeric polymerase, Tfpol, which has proven to be effective with complex samples that would be hostile to conventional PCR strategies at the tested concentrations. Most importantly, TFpol supports multiplexed LAMP amplifications which have been infrequently demonstrated. These capabilities, taken together, enable features that are contemporary in high-throughput laboratory testing but more challenging in point-of care diagnostics.

Multiplexed LAMP reactions with the ability to differentiate individual products by target specific probes enable two key aspects of robust NAAT testing: internal amplification controls and multiple target redundancy. IACs are widely accepted as a means of assuring the sample could detect a positive result in the event of a negative outcome by demonstrating the reaction chemistry was viable and not inhibited by sample contaminants or is otherwise compromised. In the context of LAMP amplification, internal controls can impair successful target detection; the resource demands of a successful LAMP mean co-amplification of multiple products with varying inputs often lead to the competitive inhibition of slower assays or lower concentration of target (FIGS. 3B and 3C). Presumably, this can be attributed to resource depletion of limiting reagents in the reaction mix. To address this resource competition, we devised a shared-Primer internal control strategy where the performance of the IAC has been intentionally impaired by using a reduced primer set. The delayed time-to-detection of the IAC can then be further controlled by adjusting the concentration of control template, ensuring reduced competition with the target amplification. A similar principle applies to the redundant SARS-CoV-2 target assays; only one target is expected to amplify to detection in a typical reaction because of competition between them.

The single-pot multiple target redundancy is a defining feature of this assay design despite the grouped signal. Pathogen genetic variability is an important failure mode for nucleic acid amplification tests; a single nucleotide point mutation (SNP) can result in underperformance of a LAMP or PCR reaction. As the COVID-19 pandemic progresses, the virus will continue to accumulate mutations and diversify, posing a challenge to NAATs used for diagnosis. An alignment of publicly available SARS-CoV-2 genomes at the time of writing reveals multiple genomes with known mutations in the primer footprints of the CDC PCR designs and a range of other published assays, suggesting that mutations are an existential problem. Targeting multiple unique regions in the virus' genome ensures mutations that would otherwise render a singleplex test ineffective are still detectable. As a potential benefit, preventing diagnostic evasion in this way may reduce selective pressure due to intervention and treatment at individual loci and have a long-term impact. The likelihood of mutations rendering all three assays ineffective simultaneously is lower than for a single assay. Our own analysis found a difference in perfect coverage. This principle is often incorporated commercially available conventional Laboratory based NAATs so this capability represents a convergence of state-of-the-art diagnostic methods and POC diagnostic capabilities.

While illustrative embodiments have been illustrated and described, it will be appreciated that various changes can be made therein without departing from the spirit and scope of the invention.

1. A kit comprising:
a loop primer nucleic acid molecule configured for loop-mediated isothermal amplification (LAMP), the loop primer nucleic acid molecule comprising:
a targeting sequence complementary to a target portion of a target nucleic acid sequence; and
an adapter sequence;
a displacement nucleic acid probe comprising:
a fluorophore adapter sequence; and
the adapter sequence; and
a fluorophore adapter complement nucleic acid molecule complementary to the fluorophore adapter sequence,
wherein the fluorophore adapter sequence or the fluorophore adapter complement nucleic acid molecule is coupled to a fluorophore.

2. The kit of claim 1, wherein whichever of the fluorophore adapter sequence or the fluorophore adapter complement nucleic acid molecule is not coupled to the fluorophore is coupled to a quencher configured to quench fluorescence of the fluorophore.

3. The kit of claim 1, wherein whichever of the fluorophore adapter sequence or the fluorophore adapter complement nucleic acid molecule is not coupled to the fluorophore is coupled to a second fluorophore configured to receive energy from the fluorophore by Förster resonance energy transfer.

4. The kit of claim 1, wherein the fluorophore adapter sequence is configured to form a hairpin structure and further comprises a quencher positioned to be proximal to the fluorophore when the fluorophore adapter sequence is in the hairpin structure and configured to quench fluorescence of the fluorophore.

5. The kit of claim 1, further comprising a second loop primer nucleic acid molecule configured for LAMP, the second loop primer nucleic acid molecule comprising:
a second targeting sequence complementary to a second target nucleic acid sequence; and
the adapter sequence.

6. (canceled)

7. The kit of claim 1 further comprising:
a second loop primer nucleic acid molecule complementary to a second portion of the target nucleic acid sequence, wherein the second portion of the target nucleic acid sequence is different than the target portion of the target nucleic acid sequence.

8. The kit of claim 1 further comprising:
a forward outer primer nucleic acid molecule complementary to an upstream portion of the target nucleic acid sequence, wherein the upstream portion is upstream of the target portion of the target nucleic acid sequence; and
a backwards outer primer nucleic acid molecule complementary to a downstream portion of the target nucleic acid sequence, wherein the downstream portion is downstream of the target portion.

9. The kit of claim 1 further comprising:
a forward inner primer nucleic acid molecule complementary to a second upstream portion of the target nucleic acid sequence, wherein the forward inner primer nucleic acid molecule further comprises a loop-forming portion complementary to a second downstream portion of the target nucleic acid sequence, wherein the second downstream portion is downstream of the downstream portion; and
a backward inner primer complementary to a third downstream portion of the target nucleic acid sequence.

10. The kit of claim 1 further comprising internal amplification control primer nucleic acid molecules comprising:
a control targeting sequence complementary to a control portion of a control target nucleic acid sequence; and
a control adapter sequence;
a control displacement nucleic acid probe comprising:
a control fluorophore adapter sequence; and
the control adapter sequence; and
a control fluorophore adapter complement nucleic acid molecule complementary to the control fluorophore adapter sequence,
wherein the control fluorophore adapter sequence or the control fluorophore adapter complement nucleic acid molecule is coupled to a control fluorophore.

11. (canceled)

12. The kit of claim 1 further comprising a polymerase.

13. The kit of claim 12, wherein the polymerase comprises an amino acid sequence at least 55% identical to SEQ ID NO. 25.

14. (canceled)

15. A method of detecting a presence or absence of a target nucleic acid sequence in a sample, the method comprising:
contacting the sample with reagents comprising:
a loop primer nucleic acid molecule configured for LAMP, the loop primer nucleic acid molecule comprising:
a targeting sequence complementary to a target portion of a target nucleic acid sequence; and
an adapter sequence;
a displacement nucleic acid probe comprising:
a fluorophore adapter sequence; and
the adapter sequence; and
a fluorophore adapter complement nucleic acid molecule complementary to the fluorophore adapter sequence,
wherein the fluorophore adapter sequence or the fluorophore adapter complement nucleic acid molecule is coupled to a fluorophore
maintaining the sample and the reagents under conditions and for a time sufficient to amplify nucleic acid molecules comprising the target nucleic acid sequence; and
detecting the presence or absence of fluorescence from the fluorophore.

16-20. (canceled)

22. A polypeptide comprising an amino acid sequence at least 55% identical to SEQ ID NO. 25.

23-31. (canceled)

32. A nucleic acid encoding the polypeptide of claim 22.

33. A nucleic acid expression vector comprising the nucleic acid of claim 32.

34. A recombinant host cell comprising the nucleic acid expression claim 33.

35. A method of amplifying a nucleic acid molecule, the method comprising:
contacting the nucleic acid molecule with a polypeptide according to claim 22 under conditions and for a time sufficient to amplify the nucleic acid molecule.

36. A system for detection of amplification of a nucleic acid molecule in a sample, the system comprising:
a thermal subsystem comprising:
a thermally conductive heat block defining a chamber shaped to carry a sample holder configured to carry the sample; a first aperture disposed on a first side of the heat block and positioned to emit first signal light from within the sample holder disposed in the chamber; and a second aperture disposed on a second side of the heat block opposite the first side and positioned to emit second signal light from within the sample holder disposed in the chamber; and
a heat source thermally coupled to the heat block; and
an optical subsystem comprising:
a light source configured to emit excitation light into the chamber;
a first photodetector positioned to receive the first signal light from within the sample holder through the first aperture; and
a second photodetector positioned to receive the second signal light from within the sample holder through the second aperture.

37-51. (canceled)

52. A method of detecting amplification of a nucleic acid molecule in a sample, the method comprising:
heating a thermally conductive heat block with a heat source thermally coupled to the heat block, wherein the heat block defines a chamber shaped to carry a sample holder carrying the sample; a first aperture disposed on a first side of the heat block and positioned to emit first signal light from within the sample holder disposed in the chamber; and a second aperture disposed on a second side of the heat block opposite the first side and positioned to emit second signal light from within the sample holder disposed in the chamber;
emitting, with the light source, the excitation light into the sample holder;
generating a first sample signal with a first photodetector based on the first signal light received by the first photodetector through the first aperture; and
generating a second sample signal with a second photodetector based on the second signal light received by the second photodetector through the second aperture.

53-55. (canceled)

56. A non-transitory, machine-readable storage medium having instructions stored thereon, which when executed by a processing system, cause the processing system to perform operations including:
heating a thermally conductive heat block with a heat source thermally coupled to the heat block, wherein the heat block defines a chamber shaped to carry a sample holder carrying a sample; a first aperture disposed on a first side of the heat block and positioned to emit first signal light from within the sample holder; and a second aperture disposed on a second side of the heat block opposite the first side and positioned to emit second signal light from within the sample holder;
emitting, with a light source, excitation light into the sample holder disposed in the chamber;
generating a first sample signal with a first photodetector based on the first signal light received by the first photodetector through the first aperture; and
generating a second sample signal with a second photodetector based on the second signal light received by the second photodetector through the second aperture.


SEQUENCE LISTING (29 sequences)

SEQ ID NO: 1 - DNA, 40 bp, Artificial Sequence
  Synthetic
ccactgcgttctccattcttttccccgcattacgtttggt

SEQ ID NO: 2 - DNA, 43 bp, Artificial Sequence
  Synthetic
gcgatcaaaacaacgtcggttattgccatgttgagtgagagcg

SEQ ID NO: 3 - DNA, 21 bp, Artificial Sequence
  Synthetic
tggttactgccagttgaatct

SEQ ID NO: 4 - DNA, 52 bp, Artificial Sequence
  Synthetic
accaacacctcacatcacacataataggtttacccaataatactgcgtcttg

SEQ ID NO: 5 - DNA, 18 bp, Artificial Sequence
  Synthetic
tggaccccaaaatcagcg

SEQ ID NO: 6 - DNA, 22 bp, Artificial Sequence
  Synthetic
atctggactgctattggtgtta

SEQ ID NO: 7 - DNA, 40 bp, Artificial Sequence
  Synthetic
cagcttctggcccagttcctgtggtggtgacggtaaaatg

SEQ ID NO: 8 - DNA, 48 bp, Artificial Sequence
  Synthetic
cttccctatggtgctaacaaagtccaatgtgatcttttggtgtattca

SEQ ID NO: 9 - DNA, 24 bp, Artificial Sequence
  Synthetic
gtagtagaaataccatcttggact

SEQ ID NO: 10 - DNA, 48 bp, Artificial Sequence
  Synthetic
accaacacctcacatcacacataataatatgggttgcaactgagggag

SEQ ID NO: 11 - DNA, 21 bp, Artificial Sequence
  Synthetic
ctactaccgaagagctaccag

SEQ ID NO: 12 - DNA, 21 bp, Artificial Sequence
  Synthetic
gcagcattgttagcaggattg

SEQ ID NO: 13 - DNA, 39 bp, Artificial Sequence
  Synthetic
tgtgtaggtcaaccacgttctgcttcagcgttcttcgga

SEQ ID NO: 14 - DNA, 53 bp, Artificial Sequence
  Synthetic
gtgccatcaaattggatgacaaaggttttgtatgcgtcaatatgcttattcag

SEQ ID NO: 15 - DNA, 45 bp, Artificial Sequence
  Synthetic
accaacacctcacatcacacataatatccatgccaatgcgcgaca

SEQ ID NO: 16 - DNA, 23 bp, Artificial Sequence
  Synthetic
ccaaatttcaaagatcaagtcat

SEQ ID NO: 17 - DNA, 22 bp, Artificial Sequence
  Synthetic
gaccaggaactaatcagacaag

SEQ ID NO: 18 - DNA, 21 bp, Artificial Sequence
  Synthetic
gcttgagtttcatcagccttc

SEQ ID NO: 19 - DNA, 47 bp, Artificial Sequence
  Synthetic
accacacctaccaccactaataactaactccagccatcctcaccatc

SEQ ID NO: 20 - DNA, 60 bp, Artificial Sequence
  Synthetic
ccatcagcaccaagactacccacctcgccaccaaaccaacacctcacatcacacataata

SEQ ID NO: 21 - DNA, 34 bp, Artificial Sequence
  Synthetic
ttggtggcgaggtgggtagtcttggtgctgatgg

SEQ ID NO: 22 - DNA, 61 bp, Artificial Sequence
  Synthetic
cctgaccacttccgaacccaaccacctacgacagaccacacctaccaccactaataactaa

SEQ ID NO: 23 - DNA, 34 bp, Artificial Sequence
  Synthetic
ctgtcgtaggtggttgggttcggaagtggtcagg

SEQ ID NO: 24 - DNA, 200 bp, Artificial Sequence
  Synthetic
aatggaccccaaaatcagcgaaatgcaccccgcattacgtttggtggaccctctggagtcaatgggtggtgccagaatggagaacgcagtggggcgcgatcaaaacaacgtcggccccaagttgatctccagccatcctcaccatcgttcaccgctctcactcaacatggcaagaattaacaccaatagcagtccagatg

SEQ ID NO: 25 - protein, 540 aa, Artificial Sequence
  Synthetic
PLEEAPWPPPEGAFVGFVLSRPEPMWAELKALAACRDGRVHRAEDPLAGLGDLEEVRGLLAKDLAVLALREGLDLAPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTINFGIVYGMSPYGLAKELKIGRREAKAFIERYFERYPGVKRYMEQIVAEAREKGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGIGEDWLSAKG

SEQ ID NO: 26 - protein, 559 aa, Artificial Sequence
  Synthetic
MKSSHHHHHHENLYFQSNAPLEEAPWPPPEGAFVGFVLSRPEPMWAELKALAACRDGRVHRAEDPLAGLGDLEEVRGLLAKDLAVLALREGLDLAPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTINFGIVYGMSPYGLAKELKIGRREAKAFIERYFERYPGVKRYMEQIVAEAREKGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGIGEDWLSAKG

SEQ ID NO: 27 - protein, 550 aa, Artificial Sequence
  Synthetic
MKSSHHHHHHPLEEAPWPPPEGAFVGFVLSRPEPMWAELKALAACRDGRVHRAEDPLAGLGDLEEVRGLLAKDLAVLALREGLDLAPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTINFGIVYGMSPYGLAKELKIGRREAKAFIERYFERYPGVKRYMEQIVAEAREKGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGIGEDWLSAKG

SEQ ID NO: 28 - DNA, 26 bp, Artificial Sequence
  Synthetic
accaacacctcacatcacacataata

SEQ ID NO: 29 - DNA, 27 bp, Artificial Sequence
  Synthetic
accacacctaccaccactaataactaa